SEQUENCE LISTING (10 sequences)

SEQ ID NO: 1 - DNA, 24 bp, synthetic construct
  source 1..24: mol_type = other DNA
aaccgggaggccagctaaagaaca

SEQ ID NO: 2 - DNA, 23 bp, synthetic construct
  source 1..23: mol_type = other DNA
tgggcctacagaacagacagtgc

SEQ ID NO: 3 - DNA, 20 bp, synthetic construct
  source 1..20: mol_type = other DNA
gagaacctggagaagcagct

SEQ ID NO: 4 - DNA, 18 bp, synthetic construct
  source 1..18: mol_type = other DNA
ccgcggttggtccagtag

SEQ ID NO: 5 - DNA, 20 bp, synthetic construct
  source 1..20: mol_type = other DNA
gtacgccgacctcatcacca

SEQ ID NO: 6 - DNA, 20 bp, synthetic construct
  source 1..20: mol_type = other DNA
tgctgtcgcccttatccttg

SEQ ID NO: 7 - DNA, 19 bp, synthetic construct
  source 1..19: mol_type = other DNA
tcgcctcctggcgggctta

SEQ ID NO: 8 - DNA, 20 bp, synthetic construct
  source 1..20: mol_type = other DNA
acggcggtgctagcctgaga

SEQ ID NO: 9 - DNA, 48 bp, synthetic construct
  source 1..48: mol_type = other DNA
tcgtcggcagcgtcagatgtataagagacagcctacgggnggcwgcag

SEQ ID NO: 10 - DNA, 45 bp, synthetic construct
  source 1..45: mol_type = other DNA
gtctcgtgggctcggagatgtgtataagaccagagtatcactach

(19) United States
(12) Patent Application Publication
(10) Pub. No.: US 2025/0017996 A1
(43) Pub. Date: Jan. 16, 2025

(54) COMPOSITION, FOR PREVENTING, ALLEVIATING, OR TREATING MUSCLE WEAKNESS-RELATED DISEASE, COMPRISING CELERY SEED EXTRACT AS ACTIVE INGREDIENT

(71) Applicant: KYUNGPOOK NATIONAL UNIVERSITY INDUSTRY-ACADEMIC COOPERATION FOUNDATION, Daegu (KR)

(72) Inventors: Eun-Young KWON, Daegu (KR); Youngji HAN, Daegu (KR); Ji-Won KIM, Daegu (KR); Hae-Jin PARK, Gyeongsangbuk-do (KR)

(21) Appl. No.: 18/690,487

(22) PCT Filed: Jul. 26, 2022
(86) PCT No.: PCT/KR2022/010966

§ 371 Date: Oct. 4, 2024

(30) Foreign Application Priority Data
Sep. 10, 2021 (KR) 10-2021-0121287
Jul. 12, 2022 (KR) 10-2022-0085416

(51) Int. Cl. A61K 36/23; A61P 21/00

(52) U.S. Cl. CPC A61K 36/23; A61P 21/00; A61K 2236/33

(57) ABSTRACT

The present invention relates to a composition, for preventing, relieving, or treating a muscle weakness-related disease, comprising a celery seed extract. The celery seed extract according to the present invention causes a few or no side effects by using a natural product, may increase muscle mass and muscle strength to not only prevent muscle weakness but also inhibit weight gain and body fat increase, and may reduce serum lipid profiles and hepatotoxicity markers (GOT and GPT), and thus is expected to be utilized for preventing, relieving, or treating muscle weakness-related diseases, metabolic diseases, liver diseases, or the like.

### TECHNICAL FIELD

The present invention relates to a composition for preventing, alleviating or treating diseases related to muscle weakness, which includes celery seed extract as an active ingredient.

The present invention claims priority based on Korean Patent Application No. 10-2021-0121287 filed on Sep. 10, 2021 and Korean Patent Application No. 10-2022-0085416 filed on Jul. 12, 2022, and all contents disclosed in the specification and drawings of the above applications are incorporated into this application.

### BACKGROUND ART

Skeletal muscle is the largest organ in the human body, accounting for 40 to 50% of the total body weight, and has an important role in various metabolic functions in the body, including energy homeostasis and heat generation. As the human body ages, redistribution of body fat and body proteins occurs as a result of changes in components thereof. At about age 50, the rate of protein synthesis in muscle cells slows down compared to the rate of decomposition, and muscles begin to degenerate rapidly.

Sarcopenia refers to a state in which about 13 to 24% of one's usual body mass is reduced, and represents a decrease in protein content, fiber diameter, muscle strength production, and fatigue resistance. Sarcopenia is caused by a variety of causes, including sepsis, cancer, renal failure, excess glucocorticoids, denervation, muscle underuse, obesity, and the aging process. Mostly, it can be attributed to the gradual decrease in the quantity and quality of skeletal muscle that occurs as aging progresses.

On the other hand, sarcopenic obesity, in which the amount of body fat increases along with the decrease in muscle mass or strength with aging has become a problem. Obesity and sarcopenia in the elderly are presumed to act synergistically to exacerbate the risk of functional and metabolic disorders as well as the risk of death, and are suggested to have a strong interaction with each other from an etiological point of view. Therefore, in the case of an overweight person, an obese person, or a person with normal weight but high body fat, metabolic diseases such as diabetes and hypertension can be prevented and treated by increasing muscle mass and reducing body fat. Increased muscle mass may increase basal metabolic energy, enabling efficient diet without yo-yo phenomenon. Therefore, exercise, diet, and ergogenic aids are used to increase muscle mass, however, the commercially available ergogenic aids contain a chemical compound and are accompanied by fatal side effects.

Accordingly, the inventors of the present invention have tried to develop a therapeutic agent related to muscle weakness without side effects while being highly effective. As a result, the present invention was completed by confirming that celery seed extract increases muscle mass and muscle strength in obese or aging mice.

### DISCLOSURE

### Technical Problem

As a result of efforts to develop active substances derived from natural products capable of preventing, improving or treating muscle weakness-related diseases, the preset inventors confirmed that celery seed extract has excellent effects in increasing muscle mass and improving muscle metabolism in obese or aging mice, thereby completing the invention.

Accordingly, an object of the present invention is to provide a pharmaceutical composition for preventing or treating muscle weakness-related diseases, which comprises celery seed extract as an active ingredient.

Further, another object of the present invention is to provide a food composition for preventing or alleviating muscle weakness-related diseases, which comprises celery seed extract as an active ingredient.

In addition, a further object of the present invention is to provide a feed or feed additive for strengthening muscle strength, which comprises celery seed extract as an active ingredient.

However, the technical tasks to be achieved by the present invention are not limited to the above-mentioned problems, and other problems not mentioned will also be clearly understood by those skilled in the art from the description below.

### Technical Solution

In order to solve the above problems, the present invention provides a pharmaceutical composition for preventing or treating muscle weakness-related diseases, which comprises celery seed extract as an active ingredient.

Further, the present invention provides a food composition for preventing or alleviating muscle weakness-related diseases, which comprises celery seed extract as an active ingredient.

Further, the present invention provides a composition for strengthening muscle strength, which comprises celery seed extract as an active ingredient.

In addition, the present invention provides a feed or feed additive for strengthening muscle strength, which comprises celery seed extract as an active ingredient.

In one embodiment of the present invention, the celery seed extract may be an extract obtained by one or more solvents selected from the group consisting of water, alcohol having 1 to 6 carbon atoms, acetone, ether, benzene, chloroform, ethyl acetate, methylene chloride, hexane, cyclohexane, petroleum ether, subcritical fluid, and supercritical fluid, but is not limited thereto.

In another embodiment of the present invention, the muscle weakness-related disease may be one or more selected from the group consisting of sarcopenia, muscular atrophy, muscle dystrophy, and cardiac atrophy, but is not limited thereto.

In another embodiment of the present invention, the sarcopenia may be age-related sarcopenia or obesity sarcopenia, but is not limited thereto.

In another embodiment of the present invention, the composition may have one or more of the following characteristics, but is not limited thereto:
(a) increase in muscle mass;
(b) inhibition of muscle mass loss;
(c) increased muscle strength
(d) inhibition of increase in muscle tissue lipid content; or
(e) inhibition of muscular fibrosis.

In another embodiment of the present invention, the composition may further have one or more of the following characteristics, but is not limited thereto:
(a) inhibition of body weight and body fat gain;
(b) inhibition of fibrosis in liver or adipose tissue;
(c) inhibition of plasma lipid concentration increase;
(d) inhibition of increase in liver tissue lipid content;
(e) inhibition of increase in plasma hepatotoxicity index;
(f) inhibition of lipid peroxide increase and increase of antioxidant activity; or
(g) improvement of intestinal microbial imbalance.

Further, the present invention provides a method for prevention or treatment of a disease related to muscle weakness, metabolic disease; or liver disease, which comprises administering a composition comprising celery seed extract to a subject in need thereof.

Further, the present invention provides a use of a composition comprising celery seed extract, for prevention or treatment of muscular weakness-related diseases; metabolic disease; or liver disease.

Further, the present invention provides a use of celery seed extract for production of a drug for treating muscle weakness-related diseases; metabolic disease; or liver disease.

Further, the present invention provides a composition for preventing, alleviating, or treating metabolic diseases, which comprises celery seed extract as an active ingredient.

Further, the present invention provides a composition for preventing, alleviating, or treating liver disease, which comprises celery seed extract as an active ingredient.

Further, the present invention provides a muscle strengthening method, which comprises administering a composition comprising celery seed extract to a subject in need thereof.

Further, the present invention provides a muscle strengthening use of a composition comprising celery seed extract.

Further, the present invention provides a use of celery seed extract for production of a drug for reinforcing muscle strength.

### Advantageous Effects

The celery seed extract according to the present invention has no or few side effects since a natural product is used, and can increase muscle mass and strength, so that it not only prevents muscle weakness but also suppresses the increase in body weight and body fat; and improves blood lipid concentration and liver toxicity index (GOT and GPT). Therefore, it is expected that the celery seed extract may be useful for preventing, improving, or treating diseases related to muscle weakness, metabolic diseases, or liver diseases.

### DESCRIPTION OF THE DRAWINGS

FIG. 1 is a diagram schematically illustrating an experimental design using an obesity-induced mouse model in order to assess the effect of celery seed extract on obese muscle loss.

FIG. 2a illustrates the weights of gastrocnemius, quadriceps, tibialis anterior, kidney and liver per 100 g of body weight, which were extracted and weighed after feeding the normal diet group (ND), the high fat diet group (HFD), and the celery seed extract added group (CS) mice for 20 weeks, respectively (HFD vs. ND: *p<0.05, **p<0.01, ***p<0.001; HFD vs. CS: #p<0.05, ##p<0.01, ###p<0.001, the same below).

FIG. 2b illustrates the results of comparison of weight changes (left) and weight gain inhibitory efficacy (right) according to breeding period by measuring the body weight of the normal diet group (ND), the high fat diet group (HFD), and the celery seed extract added group (CS) mice, respectively, at weekly intervals for 20 weeks.

FIG. 2c illustrates the results of comparison of food intake, energy intake and dietary efficiency (FER) measurements in regard to the normal diet group (ND), the high fat diet group (HFD), and the celery seed extract added group (CS) mice.

FIG. 2d illustrates the weight of white adipose tissue (White Adipose Tissue; WAT) per 100 g of body weight, which was extracted and weighed for each part after feeding the normal diet group (ND), the high fat diet group (HFD), and the celery seed extract added group (CS) mice for 20 weeks, respectively (epididymal, perirenal, retroperitoneum, mesentric, visceral, Subcutaneous, interscapular WAT: interscapular white fat, interscapular BAT: interscapular liver brown fat).

FIG. 3a illustrates the morphological changes in muscle tissue at 20 weeks, which were observed after feeding the normal diet group (ND), the high fat diet group (HFD), and the celery seed extract added group (CS) mice for 20 weeks, respectively (upper: H&E staining, lower: sirius red staining). Scale bar=100 μm.

FIG. 3b illustrates the morphological changes in epididymal white adipose tissue at 20 weeks, which were observed after feeding the normal diet group (ND), the high fat diet group (HFD), and the celery seed extract added group (CS) mice for 20 weeks, respectively (upper: H&E staining, lower: Masson's trichrome staining). Scale bar=100 μm.

FIG. 3c illustrates the morphological changes in liver tissue at 20 weeks, which were observed after feeding the normal diet group (ND), the high fat diet group (HFD), and the celery seed extract added group (CS) mice for 20 weeks, respectively (upper: H&E staining, lower: Masson's trichrome staining). Scale bar=100 μm.

FIG. 4 illustrates the measured results of thigh thickness at 4 weeks and 19 weeks (upper) and tensile strength at 20 weeks (lower) in regard to the normal diet group (ND), the high fat diet group (HFD), and the celery seed extract added group (CS) mice, respectively.

FIG. 5a illustrates the measured results of lipid content of muscle tissue in regard to the normal diet group (ND), the high fat diet group (HFD), and the celery seed extract added group (CS) mice, respectively (FA: free fatty acid, TG: neutral fat, CHOL: cholesterol).

FIG. 5b illustrates the measured results of lipid content of liver tissue and the activity of lipid metabolism enzyme (PAP) in regard to the normal diet (ND), the high fat diet (HFD), and the celery seed extract added group (CS) mice, respectively (FA: free fatty acids, TG: triglycerides, CHOL: cholesterol).

FIG. 6 illustrates the results of comparison of changes in expression of muscle cell growth-related proteins in regard to the normal diet group (ND), the high fat diet group (HFD), and the celery seed extract added group (CS) mice.

FIG. 7 illustrates the results of comparison of plasma triglyceride (left) and plasma total cholesterol (right) measurements for each week in regard to the normal diet group (ND), the high fat diet group (HFD), and the celery seed extract added group (CS) mice.

FIG. 8 illustrates the results of comparison of plasma glutamic oxaloacetic transaminase (GOT) and glutamic pyruvic transaminase (GPT) measurements in regard to the normal diet group (ND), the high fat diet group (HFD), and the celery seed extract added group (CS) mice.

FIG. 9a illustrates the results of comparison of paraoxonase (PON) measurements in the plasma and liver tissues in regard to the normal diet group (ND), the high fat diet group (HFD), and the celery seed extract added group (CS) mice.

FIG. 9b illustrates the results of comparison of lipid peroxide (TBARS) measurements in red blood cells and liver tissues in regard to the normal diet group (ND), the high fat diet group (HFD), and the celery seed extract added group (CS) mice.

FIG. 9c illustrates the results of comparison of glutathione (GSH) measurements in red blood cells and liver tissues in regard to the normal diet group (ND), the high fat diet group (HFD), and the celery seed extract added group (CS) mice.

FIG. 10 is a diagram schematically illustrating an experimental design using an aged mouse model in order to assess the effect of celery seed extract on age-related muscle loss.

FIG. 11 illustrates the results of confirming weight changes according to the breeding period by measuring the body weight of the young mouse group (YC), the aged mouse group (NC), and the celery seed extract added group (CS), respectively, at weekly intervals for 12 weeks (YC vs. NC: *p<0.05, **p<0.01, ***p<0.001; NC vs. CS: &p<0.05, &&p<0.01, &&&p<0.001, the same below).

FIG. 12 illustrates the results of comparison of food intake and food efficiency (FER) measurements in regard to the young mouse group (YC), the aged mouse group (NC), and the celery seed extract added group (CS).

FIG. 13 illustrates the weight of white adipose tissue (WAT) per 100 g of body weight, which was extracted and weighed for each part after feeding the young mouse group (YC), the aged mouse group (NC), and the celery seed extract added group (CS) with the experimental diet for 12 weeks, respectively (epididymal, perirenal, retroperitoneum, mesentric, visceral, subcutaneous, interscapular WAT: interscapular white adipose tissue, interscapular BAT: interscapular brown fat).

FIG. 14a illustrates the weights of gastrocnemius, quadriceps and tibialis per 100 g of body weight, which were extracted and weighed after feeding the young mouse group (YC), the aged mouse group (NC), and the celery seed extract added group (CS) with the experimental diet for 12 weeks, respectively.

FIG. 14b illustrates the measured results of tensile strength at 12 weeks in regard to the young mouse group (YC), the aged mouse group (NC), and the celery seed extract added group (CS), respectively.

FIG. 14c illustrates the measured results of thigh thickness at 4 weeks and 12 weeks in regard to the young mouse group (YC), the aged mouse group (NC), and the celery seed extract added group (CS), respectively.

FIG. 15 illustrates the measured results of lipid content in muscle tissues in regard to the young mouse group (YC), the aged mouse group (NC), and the celery seed extract added group (CS), respectively (FA: free fatty acid, TG: neutral fat, CHOL: cholesterol).

FIG. 16 illustrates the morphological changes in muscle tissue at 12 weeks, which were observed after feeding the young mouse group (YC), the aged mouse group (NC), and the celery seed extract added group (CS) with the experimental diet for 12 weeks, respectively (upper: H&E staining, lower: sirius red staining). Scale bar=100 μm.

FIG. 17 illustrates the results of confirming the expression of IGF-1 and Myostatin by immunochemical analysis after feeding the young mouse group (YC), the aged mouse group (NC), and the celery seed extract added group (CS) with the experimental diet for 12 weeks, respectively. Scale bar=100 μm.

FIG. 18 illustrates the results of confirming the expression of muscle atrophy-related genes (FoxO1, FoxO3, Atrogin, and MuRF1) after feeding the young mouse group (YC), the aged mouse group (NC), and the celery seed extract added group (CS) with the experimental diet for 12 weeks, respectively.

FIG. 19 illustrates the results of confirming the expression of muscle fiber proteins (Igf-1R, FoxO1, and sirt3) after feeding the young mouse group (YC), the aged mouse group (NC), and the celery seed extract added group (CS) with the experimental diet for 12 weeks, respectively.

FIG. 20 illustrates the results of examining glucose tolerance after feeding the young mouse group (YC), the aged mouse group (NC), and the celery seed extract added group (CS) with the experimental diet for 12 weeks, respectively.

FIG. 21a illustrates the weight of liver per 100 g of body weight, which was measured after feeding the young mouse group (YC), the aged mouse group (NC), and the celery seed extract added group (CS) with the experimental diet for 12 weeks, respectively.

FIG. 21b illustrates the measured results of liver damage index plasma GOT and GPT after feeding the young mouse group (YC), the aged mouse group (NC), and the celery seed extract added group (CS) with the experimental diet for 12 weeks, respectively.

FIG. 21c illustrates the measured results of lipid content in liver tissue after feeding the young mouse group (YC), the aged mouse group (NC), and the celery seed extract added group (CS) with the experimental diet for 12 weeks, respectively.

FIG. 22a illustrates the measured results of glutathione (GSH) content in liver tissue, red blood cells and plasma after feeding the young mouse group (YC), the aged mouse group (NC), and the celery seed extract added group (CS) with the experimental diet for 12 weeks, respectively.

FIG. 22b illustrates the measured results of superoxide dismutase (SOD) activity in liver tissue and red blood cells after feeding the young mouse group (YC), the aged mouse group (NC), and the celery seed extract added group (CS) with the experiment diet for 12 weeks, respectively.

FIG. 22c illustrates the measured results of H₂O₂ content in mitochondria, cell matrix and red blood cells after feeding the young mouse group (YC), the aged mouse group (NC), and the celery seed extract added group (CS) with the experimental diet for 12 weeks, respectively.

FIG. 23 illustrates the measured results of contents of short-chain fatty acids such acetate, propionate, and butyrate after feeding the young mouse group (YC), the aged mouse group (NC), and the celery seed extract added group (CS) with the experimental diet for 12 weeks, respectively.

FIGS. 24a and 24b illustrate the results of analyzing the intestinal microbes after feeding the young mouse group (YC), the aged mouse group (NC), and the celery seed extract added group (CS) with the experimental diet for 12 weeks, respectively, wherein FIG. 24a shows the analysis results of the intestinal microbes involved in colitis and inflammatory response, while FIG. 24b shows the analysis results of the intestinal microbes involved in obesity and short-chain fatty acid production.

### BEST MODE

As a result of efforts to develop natural product-derived active substances capable of preventing, alleviating or treating muscle weakness-related diseases, the present inventors confirmed the excellent effects of increasing muscle mass and improving muscle metabolism of celery seed extract in obese or aged mice, therefore, completed the present invention.

Accordingly, the present invention provides a pharmaceutical composition for preventing or treating muscle weakness-related diseases, comprising celery seed extract as an active ingredient.

Further, the present invention provides a composition for strengthening muscle strength comprising celery seed extract as an active ingredient.

The “celery seed” in the present invention is a seed of celery, which is a plant of the umbel family Apiaceae, has a millet size, is yellowish brown, has a celery-like fragrance and is used as a spice. The celery seed is characterized by green smell and bitter taste, and is known to be effective in anti-inflammatory, diuretic, sedative and/oraphrodisiac activity, anti-rheumatism, and arthritis.

In the present invention, the “extract” refers to an extract obtained by an extraction process of the celery seeds, a diluted or concentrated liquid of the extract, a dried product obtained by drying the extract, a crude product or a purified product of the extract, a mixture thereof, etc., that is, includes the extract itself and extracts of all formulations that can be formed using the above extract.

The method for extracting celery seeds according to the present invention is not particularly limited, and the celery seed may be extracted according to any method commonly used in the art. Non-limiting examples of the extraction method may include a hot water extraction method, an ultrasonic extraction method, a filtration method, a reflux extraction method, and the like, which may be performed alone or in combination of two or more methods.

In the present invention, the type of extraction solvents used to extract the celery seed is not particularly limited, and the celery seed may be extracted according to any conventional method known in the art for extraction of desired ingredients from natural products, that is, using a conventional solvent under normal temperature and pressure conditions. For example, the celery seed extract in the present invention may be obtained by extraction using at least one solvent selected from the group consisting of water, an organic solvent having 1 to 6 carbon atoms, and a subcritical or supercritical fluid. The organic solvent having 1 to 6 carbon atoms may be one or more selected from the group consisting of alcohol, acetone, ether, benzene, chloroform, ethyl acetate, methylene chloride, hexane, cyclohexane and petroleum ether, each of which has 1 to 6 carbon atoms, but is not limited thereto. In the present invention, the celery seed extract is preferably obtained by extraction using ethanol.

According to the present invention, the celery seed extract may be obtained by: adding 700 mL to 1.3 L, 700 mL to 1.1 L, 700 mL to 1 L, 700 mL to 900 mL, 900 mL to 1 L, 1 L to 1.1 L, 1 L to 1.3 L, 1.2 L to 1.3 L, 700 mL, 800 mL, 900 mL or 1 L of ethanol at a concentration of 40 to 95%, 40 to 85%, 40 to 75%, 40 to 65%, 40 to 55%, 40 to 50%, 40 to 47%, 40 to 45%, 40 to 43%, 40 to 41%, 50 to 55%, 55 to 65%, 65 to 75%, 68 to 75%, 70 to 75%, 73 to 75%, 65 to 73%, 65 to 70%, 65 to 68%, 75 to 85%, 85 to 95%, 88 to 95%, 90 to 95%, 90 to 92%, 90 to 91%, 40%, 50%, 60%, 65%, 68%, 69%, 70%, 71%, 72%, 75%, 78%, 80% or 90% to 70 to 130 g, 70 to 110 g, 70 to 100 g, 70 to 80 g, 80 to 130 g, 100 to 130 g, 110 to 130 g, 90 to 110 g, 95 to 105 g, 70 g, 80 g, 90 g or 100 g of celery seed powder; repeating extraction at 30 to 90° C., 30 to 70° C., 30 to 50° C., 40 to 50° C., 50 to 70° C., 55 to 75° C., 60 to 70° C., 60 to 65° C., 60 to 63° C., 70 to 90° C., 80 to 90° C., 85 to 90° C., 30° C., 40° C., 50° C., 55° C. or 60° C. for 1 hour 30 minutes to 3 hours 30 minutes, 1 hour 30 minutes to 3 hours, 1 hour 30 minutes to 2 hours, 2 hours to 2 hours 30 minutes, 2 hours 30 minutes to 3 hours, 3 hours to 3 hours 30 minutes, 2 hours, 2 hours 30 minutes, 2 hours 50 minutes or 3 hours, 1 to 5 times, 1 to 3 times, 1 to 2 times, 2 to 4 times, 4 to 5 times, 1 time, 2 times or 3 times; filtering the extracted liquid, followed by concentration under reduced pressure and lyophilization, but is not limited thereto.

The prepared extract may then be filtered or concentrated or dried to remove the solvent, and filtration, concentration and drying may be all performed. For example, a filter paper or a vacuum filter may be used for filtration, a vacuum concentrator may be used for concentration, and a lyophilization method may be used for drying, but is not limited thereto.

In the present invention, the “active ingredient” means a component that exhibits the desired activity alone or can exhibit the desired activity together with a carrier having no activity itself.

In the present invention, the “disease related to muscle weakness” means any disease in which muscle tissues or muscle cells are reduced or lost. The muscle weakness may be limited to any one muscle, one side of the body, upper limbs or lower limbs, may appear throughout the body, or may appear congenital or acquired. Further, subjective muscle weakness symptoms including muscle fatigue or muscle pain may be quantified in an objective way through a physical examination.

The muscle weakness-related diseases refer to all diseases that may occur due to muscle weakness, and may include, but are not limited to, for example, sarcopenia, amyotrophy, muscle dystrophy or cardiac atrophy.

In the present invention, “sarcopenia” refers to a condition in which the body's muscles (muscle mass and strength) are abnormally reduced or weakened due to various reasons such as aging and obesity, resulting in poor physical activity. If the symptom becomes serious, it brings obstacle and increases mortality risks. Sarcopenia has a broad range of effects throughout the body, including bones, blood vessels, nerves, liver, heart, and pancreas, not only the muscles themselves. In particular, since bones receive stress (stimulation) by muscles and thus maintain density, sarcopenia and osteoporosis have a very close correlation. Further, it is known that, when muscle is reduced, it interferes with the formation of new blood vessels and nerves, increasing the risk of cognitive decline, fatty liver, and diabetes.

In the present invention, the sarcopenia may be age-related sarcopenia or obesity sarcopenia, but is not limited thereto.

The “age-related sarcopenia” refers to a gradual decrease in muscle mass or a gradual weakening of muscle density and function due to aging, which means a state capable of directly deriving a decrease in muscle strength, resulting in a reduction in various physical functions and disability.

The “obesity sarcopenia” refers to a phenomenon in which muscle function is weakened by the deposition of fat in the muscle due to obesity to decrease a muscle mass while increasing mast cell-derived inflammatory factors, and to reduce the function of mitochondria in the muscle. When abnormalities in insulin secretion occur due to obesity, energy cannot be properly supplied to cells, which in turn may cause muscle development disorders.

The “amyotrophy” is a gradual atrophy of the muscles of the limbs, which causes progressive degeneration of motor nerve fibers and cells in the spinal cord, resulting in amyotrophic lateral sclerosis (ALS) and spinal progressive muscular atrophy (SPMA).

The “muscle dystrophy” refers to a disease in which gradual muscle atrophy and weakness occur, and pathologically means a degenerative myopathy characterized by necrosis of muscle fibers.

The “cardiac atrophy” is a condition in which the heart is atrophied by external or internal factors, and appears as symptoms in that myocardial fibers become dry and thin due to starvation, wasting disease, and frailty, causing a decrease in adipose tissue.

As used herein, the term “strengthening muscle strength” refers to enhancement of physical performance, enhancement of maximum endurance, increase in muscle mass, enhancement of muscle recovery, reduction of muscle fatigue, improvement of energy balance, or a combination thereof.

According to one embodiment of the present invention, in a high fat diet-derived sarcopenia obese mouse model, it was confirmed that the celery seed extract increases muscle mass and strength, inhibits fibrosis of muscle tissue, suppresses an increase in lipid content of muscle tissue, increases the expression of amyotrophy-related proteins PGC1α and proteins involved in protein accumulation, MyD88 and Traf6, activates Akt and Pi3k proteins involved in myocyte differentiation and inhibition of apoptosis, in addition, suppresses weight and body fat gain, inhibits fibrosis of liver and adpose tissues, suppresses an increase in lipid content of liver tissue, suppresses an increase in plasma hepatotoxicity index, inhibits increase of lipid peroxides, and increases antioxidant capacity (see Example I).

According to another embodiment of the present invention, in an aged mouse model, it was confirmed that the celery seed extract increases muscle mass and strength, inhibits fibrosis of muscle tissue, suppresses an increase in lipid content of muscle tissue, increases the expression of IGF-1 involved in the synthesis of muscle tissue, reduces the expression of myostatin involved in the degradation of muscle tissue, reduces the expression of genes related to amyotrophy, increases the expression of igf-1 R, which is involved in muscle fiber protein synthesis, reduces the expression of FoxO1, in addition, suppresses an increase in body fat, lowers blood sugar, suppresses an increase of plasma lipid concentration, suppresses an increase in plasma hepatotoxicity index, suppresses an increase in lipid content of liver tissue, increases antioxidant capacity, increases short-chain fatty acids, and improves intestinal microbial imbalance (see Example II).

Accordingly, the celery seed extract may be applied not only to the prevention, alleviation or treatment of muscle weakness-related diseases, but also to the prevention, alleviation or treatment of metabolic diseases or liver diseases.

Therefore, in the present invention, the composition according to the present invention may satisfy one or more of the following characteristics so as to prevent, alleviate or treat muscle weakness-related diseases:
(a) increase in muscle mass;
(b) inhibition of muscle mass loss;
(c) increased muscle strength;
(d) inhibition of increase in muscle tissue lipid content; or
(e) inhibition of muscular fibrosis.

Further, in the present invention, the composition according to the present invention may satisfy one or more of the following characteristics so as to prevent, alleviate or treat muscle weakness-related diseases, metabolic diseases, or liver diseases:
(a) suppression of body weight and body fat gain;
(b) inhibition of fibrosis in liver or adipose tissue;
(c) suppression of increase in plasma lipid concentration;
(d) suppression of increase in lipid content of liver tissue;
(e) suppression of increase in plasma hepatotoxicity index;
(f) inhibition of lipid peroxide increase and increase of antioxidant activity;
(g) improvement of intestinal microbial imbalance.

In the present invention, “improvement of intestinal microbial imbalance” means promoting the proliferation or growth of beneficial bacteria in the intestine and inhibiting the proliferation or growth of harmful bacteria in the intestine, while maintaining the balance between beneficial bacteria and harmful bacteria in the intestine. Microorganisms begin to inhabit the organ, and the population reaches equilibrium to produce gut microbiome, wherein individual microbes constituting the gut microbiome participate in vitamin supply, infection prevention, intestinal function, or the like, whereby it is well known that the composition of such gut microbiomes has a close relationship with constipation and the occurrence of intestinal-related diseases.

The “intestinal beneficial bacteria” may collectively refer to microorganisms that inhabit the intestines and exert beneficial effects on the human body. For example, intestinal beneficial bacteria may include probiotics. For example, the intestinal beneficial bacteria may include, but are not limited to, Bifidobacterium genus, Lactobacillus genus, Lactococcus genus, Streptococcus genus, Akkermansia genus, Faecalibacterium genus, Roseburia genus, Lachnospiraceae class, or Enterococcus genus. However, in the case of Enterococcus genus, although there are also strains classified as beneficial bacteria and antibacterial activity in the intestine along with Lactobacillus genus, some enterococci may be classified as harmful bacteria that cause diseases such as inflammation caused by vancomycin-resistant enterococci (VRE), urinary tract infections, infectious endocarditis, etc. due to high antibiotic resistance. Therefore, additional studies on probiotic potential and safety of Enterococcus are needed.

The “intestinal harmful bacteria” may collectively refer to microorganisms that live in the intestines and exert harmful effects on the human body, such as enteritis. For example, the intestinal harmful bacteria may include, but are not limited to, Escherichia coli, Fusobacterium genus, Clostridium genus, Staphylococcus genus, Desulfovibrio genus, Desulfovibrionaceae family, Erysipelatoclostridiums genus, Enterorhabdus genus, or Porphyromonas genus.

In the present invention, the “short chain fatty acid” is a major metabolite of intestinal microorganisms, and refers to fatty acids having less than 6 carbon atoms. When short-chain fatty acids are increased, the intestinal environment is acidified to activate bowel movements, may inhibit fat absorption to reduce the amount of fat in the body, and also reduce blood triglycerides, which can prevent, improve or treat metabolic diseases such as obesity. According to one embodiment of the present invention, the short-chain fatty acids in the present invention are acetic acid (acetate), propionic acid (propionate), and butyric acid (Butyrate).

In the present invention, the “obesity” refers to a state in which adipose tissue is abnormally increased due to excessive accumulation of energy in the body due to an imbalance between energy intake and consumption. Even if it seemed outwardly a normal weight, high body fat percentage may be determined as obesity. Obesity is caused by a combination of multiple causes rather than a single cause, in particular, wrong diet habits including westernized eating habits, reduced activity, emotional factors, genetic factors, etc. may become causes of the obesity. In the present invention, the obesity may be sarcopenic obesity, but is not limited thereto.

In the present invention, the “sarcopenic obesity” means a state in which the amount of body fat is increased along with a decrease in muscle mass or strength due to aging or obesity, that is, a complex form of obesity and sarcopenia due to conversion of muscle to fat. For example, it is known that, if a person with a similar body mass index has increased body fat mass and decreased muscle mass, the risk of functional limitation and metabolic disease is higher than that of another person with balanced body fat mass and muscle mass.

In the present invention, the “metabolic disease” means a condition or disease closely related to obesity or caused by obesity, for example, may be dyslipidemia; hepatotoxic diseases including drug-induced liver injury, viral liver injury, hepatitis, cirrhosis, liver cancer or hepatic coma; or fatty liver, but is not limited thereto.

In the present invention, the “liver disease” may be any one or more selected from the group consisting of non-alcoholic fatty liver diseases and liver cancer.

In the present invention, the “non-alcoholic fatty liver disease (NAFLD)” is one of the types of fatty liver that occurs when fat accumulates in the liver in a patient who has not consumed excessive alcohol, and may refer to a simple fatty liver without an inflammatory reaction, and a broad range of diseases progressed by the same, including inflammatory response of hepatocytes, liver fibrosis and liver cirrhosis. The non-alcoholic fatty liver disease may obtain good results when detected in an early stage, but if not, it may progress to non-alcoholic steatohepatitis (NASH) due to various reasons, and may further cause cirrhosis and liver cancer.

The non-alcoholic fatty liver disease is divided into primary and secondary forms depending on the cause. The primary form occurs due to hyperlipidemia, diabetes, or obesity, which are characteristic of metabolic syndrome, while the secondary form is caused by nutritional causes (rapid weight loss, starvation, intestinal bypass), diverse drugs, toxic substances (poisonous mushrooms, bacterial toxins), metabolic causes, and other factors. The incidence of nonalcoholic fatty liver disease related to diabetes and obesity, which are important features of metabolic syndrome as a primary factor, is about 50% of diabetic patients and about 76% of obese patients. In fact, it is known that the non-alcoholic fatty liver disease occurs in almost the whole of obese diabete patients.

The non-alcoholic fatty liver disease may be at least one selected from the group consisting of simple fatty liver disease, nutritional fatty liver disease, starvation fatty liver disease, obese fatty liver disease, diabetic fatty liver disease, steatohepatitis, liver fibrosis, liver cirrhosis and cirrhosis, but is not limited thereto.

The “pharmaceutical composition” according to the present invention may further include suitable carriers, excipients and diluents commonly used in the manufacture of pharmaceutical compositions. The excipient may be, for example, one or more selected from the group consisting of a diluent, a binder, a disintegrant, a lubricant, an adsorbent, a moisturizer, a film-coating material, and a controlled release additive.

The pharmaceutical composition according to the present invention maybe formulated and used in the form of external preparations such as powders, granules, sustained-release granules, enteric granules, solutions, eye drops, elsilic agents, emulsions, suspensions, spirits, troches, perfumes, limonases, tablets, sustained-release tablets, enteric tablets, sublingual tablets, hard capsules, soft capsules, sustained-release capsules, enteric capsules, pills, tinctures, soft extracts, dry extracts, fluid extracts, injections, capsules, perfusate, hard ointments, lotion, paste agent, spray, inhalant, patch, sterile injection solution, or aerosol, wherein the external preparations may have the formulations such as cream, gel, patch, spray, ointment, hard ointment, lotion, liniment, paste or cataplasma, and the like.

The carriers, excipients and diluents to be included in the pharmaceutical composition according to the present invention may include lactose, dextrose, sucrose, oligosaccharide, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate and mineral oil.

When formulated, it may be prepared using diluents or excipients such as commonly used fillers, extenders, binders, wetting agents, disintegrants, surfactants, etc.

As additives for tablets, powders, granules, capsules, pills and troches according to the present invention, excipients such as corn starch, potato starch, wheat starch, lactose, sucrose, glucose, fructose, di-mannitol, precipitated calcium carbonate, synthetic aluminum silicate, phosphoric acid, calcium monohydrogen, calcium sulfate, sodium chloride, sodium bicarbonate, purified lanolin, microcrystalline cellulose, dextrin, sodium alginate, methylcellulose, sodium carboxymethyl cellulose, kaolin, urea, colloidal silica gel, hydroxypropyl starch, hydroxypropylmethyl cellulose (HPMC) 1928, HPMC 2208, HPMC 2906, HPMC 2910, propylene glycol, casein, calcium lactate, primosel, etc.; and binders such as gelatin, arabic gum, ethanol, agar powder, cellulose phthalate acetate, carboxymethyl cellulose, calcium carboxymethyl cellulose, glucose, purified water, sodium caseinate, glycerin, stearic acid, sodium carboxymethyl cellulose, sodium methyl cellulose, methyl cellulose, microcrystalline cellulose, dextrin, hydroxycellulose, hydroxypropyl starch, hydroxymethyl cellulose, purified shellac, starch paste, hydroxypropyl cellulose, hydroxypropylmethyl cellulose, polyvinyl alcohol, polyvinyl pyrrolidone, etc. may be used. Further, disintegrants such as hydroxypropylmethyl cellulose, corn starch, agar powder, methyl cellulose, bentonite, hydroxypropyl starch, sodium carboxymethyl cellulose, sodium alginate, calcium carboxymethyl cellulose, calcium citrate, sodium lauryl sulfate, silicic anhydride, 1-hydroxypropyl cellulose, dextran, ion exchange resin, polyvinyl acetate, formaldehyde-treated casein and gelatin, alginic acid, amylose, guar gum, sodium bicarbonate, polyvinylpyrrolidone, calcium phosphate, gelled starch, arabic gumc, amylopectin, pectin, sodium polyphosphate, ethyl cellulose, white sugar, magnesium aluminum silicate, di-sorbitol solution, light anhydrous silicic acid, etc.; and lubricants such as calcium stearate, magnesium stearate, stearic acid, hydrogenated vegetable oil, talc, lycopod, kaolin, petrolatum (Vaseline), sodium stearate, cacao butter, sodium salicylate, magnesium salicylate, polyethylene glycol (PEG) 4000, PEG 6000, liquid paraffin, hydrogen added soybean oil (Lubri wax), aluminum stearate, zinc stearate, sodium lauryl sulfate, magnesium oxide, Macrogol, synthetic aluminum silicate, silicic anhydride, higher fatty acid, higher alcohol, silicone oil, paraffin oil, polyethyleneglycol fatty acid ether, starch, sodium chloride, sodium acetate, sodium oleate, dl-leucine, light anhydrous silicate may also be used.

Additives used for the liquid formulation according to the present invention may include water, diluted hydrochloric acid, diluted sulfuric acid, sodium citrate, sucrose monostearate, polyoxyethylene sorbitol fatty acid esters (tween esters), polyoxyethylene monoalkyl ethers, lanolin ethers, lanolin esters, acetic acid, hydrochloric acid, aqueous ammonia, ammonium carbonate, potassium hydroxide, sodium hydroxide, prolamine, polyvinylpyrrolidone, ethyl cellulose, sodium carboxymethyl cellulose, and the like.

For the syrup according to the present invention, a solution of white sugar, other sugars, or a sweetener may be used, and if necessary, aromatics, coloring agents, preservatives, stabilizers, suspending agents, emulsifiers, thickeners, etc. may be used.

For the emulsion according to the present invention, purified water may be used, and if necessary, emulsifiers, preservatives, stabilizers, fragrances, etc. may be used.

For the suspension agent according to the present invention, emulsifiers such as acacia, tragacantha, methylcellulose, carboxymethylcellulose, sodium carboxymethylcellulose, microcrystalline cellulose, sodium alginate, hydroxypropylmethylcellulose (HPMC), HPMC 1828, HPMC 2906, HPMC 2910, etc. may be used, and if necessary, surfactants, preservatives, stabilizers, colorants, and fragrances may be used.

The injections according to the present invention may include: solvents such as distilled water for injection, 0.9% sodium chloride injection, Ringer's (IV) injection, dextrose injection, dextrose+sodium chloride injection, PEG, lactated IV injection, ethanol, propyleneglycol, non-volatile oil-sesame oil, cottonseed oil, peanut oil, soybean oil, corn oil, ethyl oleate, isopropyl myristate, benzene benzoate, etc.; soluble aids such as sodium benzoate, sodium salicylate, sodium acetate, urea, urethane, monoethylacetamide, butazolidine, propylene glycol, tweens, amide nicotinate, hexamine, dimethylacetamide, etc.; buffers such as weak acids and salts thereof (acetic acid and sodium acetate), weak bases and salts thereof (ammonia and ammonium acetate), organic compounds, proteins, albumin, peptone, and gums; isotonic agents such as sodium chloride; stabilizers such as sodium bisulfite (NaHSO₃) carbon dioxide gas, sodium metabisulfite (Na₂S₂O₅), sodium sulfite (Na₂SO₃), nitrogen gas (N₂), ethylenediamine tetraacetic acid, etc.; sulfating agents such as sodium bisulfide 0.1%, sodium formaldehyde sulfoxylate, thiourea, ethylenediamine disodium tetraacetate, acetone sodium bisulfite, etc.; analgesics such as benzyl alcohol, chlorobutanol, procaine hydrochloride, glucose, calcium gluconate, etc.; and suspending agents such as siemesis sodium, sodium alginate, Tween 80, aluminum monostearate, etc.

For the suppository according to the present invention, bases such as cacao butter, lanolin, witapsol, polyethylene glycol, glycerogelatin, methylcellulose, carboxymethylcellulose, a mixture of stearic acid and oleic acid, subanal, cottonseed oil, peanut oil, palm oil, cacao butter+cholesterol, lecithin, Lannet Wax, glycerol monostearate, Tween or Span, Imhausen, Monolen (propyleneglycol monostearate), glycerin, Adeps Solidus, Buytyrum Tego-G-G), Cebes Pharma 16, hexalide base 95, Cotomar, Hydroxycote SP, S-70-XXA, S-70-XX75 (S-70-XX95), Hydrokote 25, Hydrokote 711, Idropostal, Massa estrarium (A, AS, B, C, D, E, I, T), Massa-MF, Masupol, Masupol-15, Neosupostal-N, Paramound-B, Suposiro (OSI, OSIX, A, B, C, D, H, L), suppository type IV (AB, B, A, BC, BBG, E, BGF, C, D, 299), Supostal (N, Es), Wecobi (W, R, S, M, Fs), testosterone triglyceride base (TG-95, MA, 57), etc. may be used.

Solid preparations for oral administration may include tablets, pills, powders, granules, capsules, etc., and these solid preparations may be prepared by mixing the extract with at least one excipient, for example, starch, calcium carbonate, sucrose, etc.), lactose or gelatin. In addition to simple excipients, lubricants such as magnesium stearate and talc may also be used.

Liquid preparations for oral administration may include suspensions, oral liquids, emulsions, syrups, etc. In addition to water and liquid paraffin, which are commonly used simple diluents, diverse excipients such as wetting agents, sweeteners, aromatics, and preservatives may be included.

Formulations for parenteral administration may include sterilized aqueous solutions, non-aqueous solvents, suspensions, emulsions, freeze-dried formulations, and suppositories. Propylene glycol, polyethylene glycol, vegetable oils such as olive oil, and injectable esters such as ethyl oleate may be used as the non-aqueous solvents and suspending agents.

The pharmaceutical composition according to the present invention is administered in a pharmaceutically effective amount. In the present invention, the “pharmaceutically effective amount” means an amount sufficient to treat a disease with a reasonable benefit/risk ratio applicable to medical treatment, and the effective dose level may be determined according to such factors including, for example, the type of patient's disease, severity, activity of the drug, sensitivity to the drug, administration time, route of administration and excretion rate, duration of treatment and drugs used concurrently, as well as other factors well known in the medical field.

The pharmaceutical composition according to the present invention may be administered as an individual therapeutic agent or in combination with other therapeutic agents, may be administered sequentially or simultaneously with conventional therapeutic agents, and may be administered single or multiple times. Considering all of the above factors, it is important to administer an amount enabling maximum effects to be attained with the minimum amount without side effects, which can be easily determined by a person skilled in the art to which the present invention belongs.

The pharmaceutical composition of the present invention may be administered to a subject through indifferent routes. All modes of administration may be envisaged, for example, the composition may be administered by oral administration, subcutaneous injection, intraperitoneal administration, intravenous injection, intramuscular injection, paraspinal space (intrathecal) injection, sublingual administration, buccal administration, intrarectal insertion, intra-vaginal insertion, ocular administration, otic administration, nasal administration, inhalation, spraying through the mouth or nose, dermal administration, transdermal administration, and the like.

The pharmaceutical composition of the present invention may be determined according to the type of drug as an active ingredient along with various relevant factors such as the disease to be treated, the route of administration, the age, sex and weight of the patient, severity of the disease, or the like.

Further, the present invention provides a method for preventing or treating muscle weakness-related diseases, which comprises administering a composition comprising celery seed extract to a subject in need thereof.

Further, the present invention provides a use of a composition comprising celery seed extract for preventing or treating muscle weakness-related diseases.

Further, the present invention provides a use of the celery seed extract for preparing a drug for treatment of muscle weakness-related diseases.

In the present invention, the “individual” means a subject in need of treatment of a disease, and more specifically, a human or non-human primate, mammals such as mouse, rat, dog, cat, horse, cow, etc.

Administration in the present invention refers to providing a given composition of the present invention to a subject by any suitable method.

In the present invention, “prevention” refers to any action that suppresses or delays the onset of a target disease, and “treatment” refers to any action that improves or advantageously changes a target disease and its associated metabolic abnormalities by administration of the pharmaceutical composition according to the present invention.

In another aspect of the present invention, the present invention provides a food composition for preventing or alleviating muscle weakness-related diseases, which comprises celery seed extract as an active ingredient.

In the present invention, “alleviation” refers to all actions that reduce the parameters related to a target disease, for example, the degree of symptoms, by administration of the composition according to the present invention. For the prevention or improvement of muscle weakness-related diseases, metabolic disease or liver disease, the health functional food composition may be used before or after the onset of the disease, simultaneously with or separately from a therapeutic drug.

When the celery seed extract of the present invention is used as a food additive, the celery seed extract may be added as it is or used together with other foods or food ingredients, and may be appropriately used according to a conventional method. A mixing amount of the active ingredient may be appropriately determined according to the purpose of use (prevention, health or therapeutic treatment). In general, when manufacturing food or beverage, the celery seed extract of the present invention may be added in an amount of 15% by weight (“wt. %”) or less, or 10 wt. % or less based on the raw material. However, in the case of long-term intake for the purpose of health and hygiene or health control, the amount may be less than the above range. In fact, since there is no problem in terms of safety, the active ingredient may be used in an amount greater than the above range.

There is no particular limitation on the type of food. Examples of foods to which the above substances can be added may include meat, sausages, bread, chocolates, candies, snacks, confectionery, pizza, ramen, other noodles, gums, dairy products including ice creams, various soups, beverages, tea, drinks, alcoholic beverages and vitamin complexes, and the like, and may include all health functional foods in a conventional sense.

The health beverage composition according to the present invention may contain various flavoring agents or natural carbohydrates as additional components, like conventional beverages. The aforementioned natural carbohydrates may be, for example, monosaccharides such as glucose and fructose, disaccharides such as maltose and sucrose, polysaccharides such as dextrins and cyclodextrins, and sugar alcohols such as xylitol, sorbitol and erythritol. As the sweetener, natural sweeteners such as thaumatin and stevia extract, or synthetic sweeteners such as saccharin and aspartame may be used. A proportion of the natural carbohydrate is generally in the range of about 0.01 to 0.20 g, or about 0.04 to 0.10 g per 100 mL of the composition of the present invention.

In addition to the above, the composition of the present invention may contain various nutrients, vitamins, electrolytes, flavors, colorants, pectic acid and its salts, alginic acid and its salts, organic acids, protective colloidal thickeners, pH adjusters, stabilizers, reservatives, glycerin, alcohols, carbonation agent used in beverages, and the like. Further, the composition of the present invention may further include fruit flesh for preparing natural fruit juice, fruit juice beverages and vegetable beverages. These components may be used independently or in combination. A proportion of these additives is not critical, but is generally selected in the range of 0.01 to 0.20 parts by weight (“wt. parts”) per 100 wt. parts of the composition of the present invention.

In the present invention, the food composition may be a health functional food composition, but is not limited thereto.

In the present invention, the “health functional food” is the same term as food for special health use (FoSHU), and means food with high medical and medical effects processed to efficiently express bioregulatory functions in addition to nutritional supply. However, the food may be prepared in various forms such as tablets, capsules, powders, granules, liquids, pills, etc. in order to obtain useful effects for preventing or improving muscle weakness-related diseases, metabolic diseases, or liver diseases.

In the present invention, the “health functional food composition” is characterized in that it includes one or more of carriers, diluents, excipients and additives, and is formulated as one selected from the group consisting of tablets, pills, powders, granules, powders, capsules and liquid formulations.

The health functional food of the present invention may be prepared by a method commonly used in the art, and may be prepared with addition of raw materials and components commonly added in the art during the preparation. In addition, unlike general drugs, there is an advantage of not involving side effects that may occur when taking a drug for a long time, since food is used as a raw material. Furthermore, the health functional food of the present invention can be excellent in portability.

Further, in another aspect of the present invention, the present invention may provide a feed or feed additive for strengthening muscle strength, which comprises celery seed extract as an active ingredient.

In the present invention, the “feed” means a substance that supplies organic or inorganic nutrients necessary for maintaining the life of an animal. The feed may contain nutrients such as energy, protein, lipid, vitamins, and minerals required by animals such as livestock, and may be vegetable feed such as grains, roots and fruits, food processing by-products, algae, fibers, oils, starches, barks, grain by-products, etc., or may be animal feed such as proteins, inorganic materials, oils, minerals, oils, and single cell proteins, etc., however is not limited thereto.

In the present invention, the “feed additive” means a substance added to the feed in order to improve productivity or health of animals, which is not particularly limited thereto, but may further include amino acids, vitamins, enzymes, and flavors, silicate agents, buffers, extractants, oligosaccharides, and the like, for growth promotion and disease prevention.

The content of the celery seed extract included in the feed or feed additive for muscle strength is not particularly limited thereto, but may range from 0.001 to 1% (w/w), preferably 0.005 to 0.9% (w/w), most preferably 0.01 to 0.5% (w/w).

### Modes of the Invention

Hereinafter, preferred embodiments are proposed to support understanding of the present invention. However, the following examples are provided to more easily understand the present invention, and the content of the present invention is not limited by the following examples.

### EXAMPLE

### Example I. Effect of Celery Seed on Obesity-induced Muscle Reduction

### Experiment Materials and Methods

### 1. Preparation of Celery Seed Extract

Celery seed powder used in the experiment was purchased from ES Food Raw Materials Co., Ltd. and used. 1 L of 70% ethanol was added to 100 g of celery seed powder as a raw material, and extraction was repeated three times for 3 hours at 60° C. The extracted liquid was filtered, concentrated under reduced pressure, and freeze-dried (or lyophilized). A celery seed extract powder was obtained, stored frozen, and then used for dietary preparation. The extraction yield was 10.02%.

### 2. Experimental Animal Models

### 2-1. Laboratory Animal

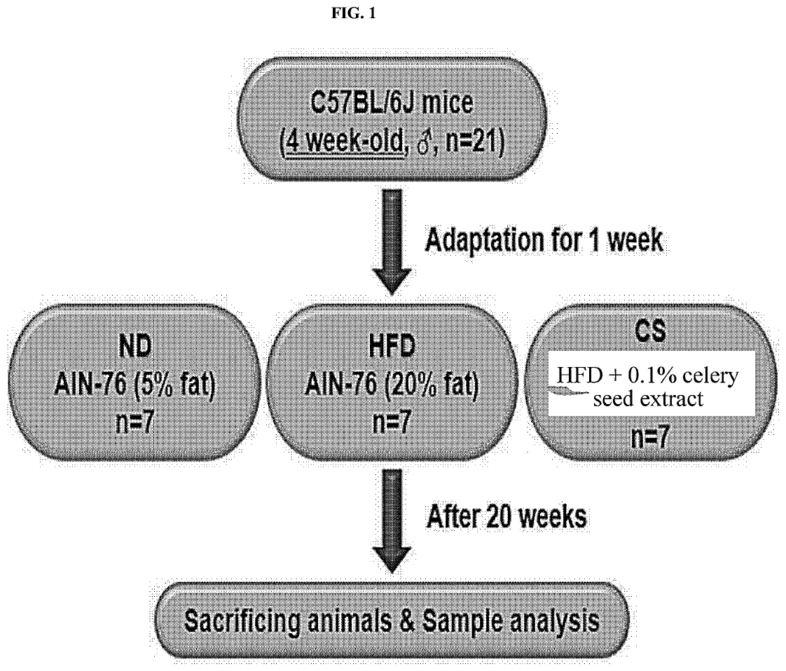

In order to confirm the effect of intake of celery seed extract, an experiment was designed and conducted as shown in the schematic diagram shown in FIG. 1 using a high fat diet-induced sarcopenic obese mouse model. Specifically, 4 week-old male C57BL/6J mice (JA BIO, Korea) were purchased from JoongA Bio and used. After adaptation by providing a diet in the form of pellets for 1 week, the mice were divided into a normal diet group (ND), a high fat diet group (HFD; 20% fat, 1% cholesterol) and a celery seed extract added group (CS, HFD+0.10% (w/w) celery seed ethanol extract) by a randomized complete block design, and fed with experimental diets and raised for 20 weeks. In the animal breeding room, constant conditions were maintained with a constant temperature (24±2° C.), constant humidity (50±5%) and a photoperiod of 12 hour intervals (AM 6:00˜ PM 18:00), and experimental diet and driving water were provided ad libitum to individual cages in which the animals were placed one by one.

### 2-2. Composition of the Experimental Diet

The experimental diet compositions of the ND group, the HFD group and the CS group are shown in Table 1 below. In the ND group, AlN-76 semisynthetic diet was prepared and fed as a normal diet group, and in the HFD group, lard and cholesterol were added to the AlN-76 diet to prepare a high fat diet containing 20% fat and 1% cholesterol and then feed to the group. The CS group was fed with the high fat diet with the addition of celery seed extract at a dose of 0.1% of the diet.

TABLE 1

| | | | CS (HFD + Celery |
|---|---|---|---|
| Ingredient (g) | ND | HFD | Seed Extract 0.1%) |
| Casein | 200.00 | 200.00 | 200.00 |
| D,L-methionine | 3.00 | 3.00 | 3.00 |
| Corn starch | 150.00 | 111.00 | 111.00 |
| Sucrose | 500.00 | 370.00 | 370.00 |
| Cellulose powder | 50.00 | 50.00 | 50.00 |
| Corn oil | 50.00 | 30.00 | 30.00 |
| Lard | — | 170.00 | 170.00 |
| Mineral Mixture (AIN-76)¹ | 35.00 | 42.00 | 42.00 |
| Vitamin Mix-(AIN-76)² | 10.00 | 12.00 | 12.00 |
| Choline bitartrate | 2.00 | 2.00 | 2.00 |
| Cholesterol | — | 10.00 | 10.00 |
| Tert-butylhydroquinone³ | 0.01 | 0.04 | 0.04 |
| Cellary Seed Extract | — | — | 1.00 |
| Total (g) | 1,000.01 | 1,000.04 | 1,001.04 |
| Total energy (kcal) | 3,850.00 | 4,524.00 | 4,524.00 |

ND, normal diet (AIN-76); HFD, high-fat diet (20% fat, 1% cholesterol); CS, celery seed (HFD + 0.1% celery seed ethanol extract)
¹AIN-76 mineral mixture (g/kg):calcium phosphate, 500; sodium chloride, 74; potassium citrate, 2220; potassium sulfate, 52; magnesium oxide, 24; manganous carbonate, 3.5; ferric citrate, 6; zinc carbonate, 1.6; cupric carbonate, 0.3; potassium iodate, 0.01; sodium selenite, 0.01; chromium potassium sulfate, 0.55; sucrose 118.03.
²AIN-76 vitamin mixture (g/kg): thiamin HCl, 0.6; riboflavin, 0.6; pyridoxine HCl, 0.7; nicotinic acid, 0.003; D-calcium pantothenate, 0.0016; folate, 0.2; D-biotin, 0.02; cyanocobalamin (vitamin B12), 0.001; retinyl palmitate premix, 0.8; DL-alpha tocopheryl acetate, premix, 20; cholecalciferol (vitamin D3), 0.0025; menaquinone (vitamin K), 0.05; antioxidant, 0.01; sucrose, finely powdered, 972.8.
Weight (g) of tert-butylhydroquinone and bioactive compounds was not included in the total weight of 1,000 g.

### 3. Sample Collection

### 3-1. Blood Sample Collection

After fasting for 12 hours every 4 weeks during the breeding period, blood lipid concentration was measured by tail blood sampling without anesthesia. After fasting for 12 hours at the end of breeding, they were anesthetized with isoflorane (5 mg/kg body weight, Baxter, USA), and blood was collected from the inferior vena cava. The collected blood was treated with heparin, centrifuged at 3,000 rpm and 4° C. for 15 minutes, and plasma was collected and stored at −70° C. until sample analysis.

### 3-2. Tissue Sample Collection

Liver, kidney, epididymal white fat, perirenal white fat, interscapular white fat, brown fat, and muscle tissues obtained by sacrificing mice after collecting blood from the abdominal inferior vena cava in the above section 3-1, were mixed with saline (0.9% saline solution). After rinsing several times in the solution, the surface water was removed and weighed, divided according to the purpose of the experiment, and rapidly cooled in liquid nitrogen and stored at −70° C. until analysis.

### 4. Measurement of Thigh Thickness and Whole-Limb Tensile Strength

During the breeding period, a thigh thickness of the mouse was measured at 4-week intervals using calipers.

After adapting to the measuring device for 3 days in order to measure the tensile force of the mice at the 19th week of feeding the experimental diet, the tensile strength was measured continuously for 5 days. Placing the mice on top of the grid of the measuring device and holding the grid with all four feet. Then, when gently and slowly pulling the tail backward while keeping a body of the mouse parallel to the grid, a maximum muscular strength by the mouse forcibly gripping the grid was recorded.

### 5. Plasma Lipid Concentration Analysis

Plasma triglyceride (TG), total cholesterol, HDL-cholesterol concentrations were measured using an enzyme kit from Asan Pharm Co., Seoul, Korea. Free fatty acid (FA) content was measured using a test solution for measuring free fatty acid (Non-Esterified Fatty Acid, NEFA kit, Shinyang Chemical) that applies the principle of color development using an enzymatic method. Plasma apolipoprotein A-1 (Apo A-1) and apolipoprotein B (apolipoprotein B; Apo B) concentrations were measured by a kit for measuring Apo A-1 and Apo B (Nitto Prefecture Co., Ltd., Tokyo, Japan).

### 6. Measurement of Tissue Lipid Content

To measure lipid content, liver and muscle tissue lipids were extracted according to the method of Folch et al. (1957), and then the extract was volatilized with nitrogen gas at 37° C. and diluted with isopropanol. For quantification, enzymatic reagent was mixed with 3 mM cholic acid as an emulsifier and 0.5% Triton X-100 to remove turbidity during color development to extract lipid components, followed by quantification which is the same as the quantification method of TG, total cholesterol and FA.

### 7. Enzyme Activity Measurement

### 7-1. Erythrocyte Enzyme Source Isolation

According to the method of McCord and Fridovich (1969), the heparinized blood was centrifuged at 3,000 rpm and 4° C. for 15 minutes to completely remove plasma and buffy coat, and then washed three times with 0.9% physiological saline. The washed red blood cells were lysed with the same amount of distilled water and used for measurement of antioxidant enzyme activity.

### 7-2. Liver Tissue Enzyme Source Isolation

For isolation of enzyme sources in liver tissue, the isolation method conducted by Hulcher et al. (1973) was partially modified and applied. After adding a buffer solution containing 0.1 M triethanolamine, 0.02 M ethylenediamine tetracetate (EDTA, pH 7.4), and 0.002 M dithiothreitol (DTT) to 0.5 g of liver tissue and homogenizing with a glass teflon homogenizer (Glascol, 099C K44, USA) in an ice-cooled state, centrifugation was performed at 3,000 rpm and 4° C. for 15 minutes, and only the supernatant was centrifuged again at 13,000 rpm and 4° C. for 15 minutes. Among them, the precipitate in a lower layer separated from the supernatant was used as a mitochondrial fraction, and the supernatant was treated using an ultracentrifuge (Beckman, Optima TLX-120, USA) for 1 hour at 32,500 rpm and 4° C. to thus obtain the cytosol fraction of the supernatant. For the microsome fraction, the same buffer solution was added to pellets separated from the cytoplasmic fraction in the supernatant, followed by ultracentrifugation at 33,000 rpm at 4° C. for 40 minutes, and then the pellets was dissolved in 1 ml buffer and stored at −70° C., and then used for analysis and protein quantification.

### 7-3. Measurement of Lipid Metabolism Enzyme Activity (Phosphatidate Phosphohydrolase)

Phosphatidate phosphohydrolase (PAP) activity was measured according to the method of Walton et al. (1985). 0.05 M Tris-HCl (pH 7.0), 1.25 mM Na₂-EDTA, 50 μl of a reaction solution with and without addition of 1 mM MgCl₂ was dissolved in a 0.9% NaCl solution, and 50 μl of a substrate containing 1 mM phosphatidate and phosphatidylcholine was added thereto. After addition, 0.1 mL of the microsome fraction was added to initiate the reaction. After reacting at 37° C. for 15 minutes, 0.1 mL of 1.8 MH₂SO₄ was added to stop the reaction, and then 0.1 mL of 0.13% sodium dodecyl sulfate (SDS) solution and 0.25 mL of each of 1.25% ascorbic acid (ascorbic acid) and 0.32% ammonium molybdate were added, followed by reacting and developing at 45° C. for 20 minutes. Thereafter, an absorbance was measured at 820 nm.

### 7-4. Measurement of Antioxidant Enzyme Activity

7-4-a. Measurement of Paraoxonase Activity

Paraoxonase (PON) activity was measured by modifying and supplementing the method of Mackness et al. (1991). As a reaction solution, 30 μl of plasma and liver tissue microsomes as enzyme sources were added to 940 μl of 0.1 M Tris-HCl buffer (pH 8.0) containing 2 mM CaCl₂), and then 30 μl of 100 mM paraoxon (O,O-diethyl-O-p-nitrophenylphosphate, Sigma Chemical Co.) as a substrate was added thereto, followed by measuring an increase in absorbance of p-nitropheol (extinction coefficient: 17,000 M⁻¹ cm⁻¹) generated at 25° C. and 405 nm for 90 seconds.

7-4-b. Glutathione (GSH) Content Measurement

Glutathione content included both oxidized and reduced GSH, and was measured by modifying and supplementing the method of Fiala et al. (1976). 0.5 g of liver tissue was added to a buffer solution containing 0.1 M triethanolamine, 0.02 M EDTA (pH 7.4), and 0.002 M DTT, and homogenized with a glass teflon homogenizer (Glascol, 099C K44, USA) in an ice-cooled state. 0.3 mL of distilled water and 0.5 mL of 4% sulfosalicylic acid were added to 0.2 mL of the homogenate, followed by centrifugation at 25,000 rpm and 4° C. for 10 minutes to obtain a supernatant. To 0.3 mL of the supernatant, 2.7 mL of 0.1 M disulfide reagent (5.5′-dithiobis+0.1 M sodiumphosphate buffer, pH 8.0) was added, followed by a reaction for 20 minutes at room temperature, and then an absorbance was measured at 412 nm.

### 8. Measurement of Red Blood Cell and Liver Tissue Lipid Peroxides (TBARS) Content

### 8-1. Measurement of Red Blood Cell Lipid Peroxide Content

Red blood cell lipid peroxide content was measured using the method of Tarladgis et al. (1964). 3 mL of 5% triochloroacetic acid (TCA) and 1 mL of 0.06 M thiobarbituric acid (TBA) were added to 50 μl of red blood cells and reacted at 80° C. for 90 minutes. After cooling to room temperature and centrifuging at 2,000 rpm and 25° C. for 15 minutes, the supernatant was taken and the absorbance was measured at 535 nm. Lipid Peroxidation (MDA) standard solution has hydrolyzed tetramethoxypropane (TMP), and an amount of TBA reactant at 267 nm was calculated as MDA extinction coefficient. That is, 1 mmol of TMP was dissolved in 100 mL of 0.01 N HCl solution, reacted at 50° C. for 60 minutes, cooled to room temperature, and TMP was hydrolyzed with MDA. MDA standard solution (1×10⁻⁴ M) was prepared by diluting 1 mL of the hydrolyzed TMP solution in 100 mL of 0.01 M Na₃PO₄ (pH 7.0) buffer. After obtaining the absorption spectrum of the MDA standard solution at 267 nm and correcting it by calculating the exact concentration from the extinction coefficient, a TBA-MDA chromopore standard curve was obtained. From this curve, the amount of the TBA reactant was calculated as the MDA extinction coefficient.

### 8-2. Measurement of Liver Tissue Lipid Peroxide Content

Liver tissue lipid peroxide content was measured as follows: a buffer solution containing 0.1 M triethanolamine, 0.02 M EDTA (pH 7.4), and 0.002 M DTT was added to 0.5 g of liver tissue using the method of Ohkawa et al. and homogenized with a glass teflon homogenizer (Glascol, 099C K44, USA) in ice-cooled state. 0.2 mL of the homogenate, 0.2 mL of 8.1% sodium dodecyl sulfate (SDS) solution, and 0.6 mL of distilled water were admixed and left at room temperature for 5 minutes, then, 1.5 mL of 20% acetate (pH 3.5) buffer and 1.5 mL of 0.8% TBA was added thereto, followed by a reaction at 95° C. for 1 hours. After the reaction, the sample was cooled to room temperature and added with 1 mL of distilled water and 5 mL of n-butanol:pyridine (15:1) solution, centrifuged at 3,000 rpm and 20° C. for 15 minutes, followed by measuring an absorbance at 532 nm. The MDA standard solution hydrolyzed TMP, and the amount of TBA reactant at 267 nm was calculated as the MDA extinction coefficient.

### 9. Morphological Analysis of Tissue

For morphological observation of tissue, parts of the liver, epididymal white fat and muscle tissues excised from mice were fixed in 10% formaldehyde solution for 24 hours, exchanged with the same solution twice, dehydrated with 2-fold ethanol, and paraffinized. After embedding in paraffin, 5 μm-thick tissue sections treated with poly-L-lysine were prepared, stained with hematoxylin eosin (H&E), and observed under an optical microscope at 200× magnification. To stain collagen and muscle fibers, liver and adipose tissue were stained with Masson's trichrome (connective tissue was stained blue, nuclei were stained dark red, and cytoplasm was stained pink), and muscle tissue was stained with sirius red (muscle fibers were stained yellow and collagen was stained red) and then observed with an optical microscope at 200× magnification.

### 10. Western Blot Measurement

0.1 g of muscle tissue was homogenized by adding 3 mm beads and 1 ml of lysis buffer (T-PER buffer, Thermo Scientific, Rockford, IL, USA), and then centrifuged at 14,000 rpm for 15 minutes to collect only the supernatant. It was transferred to anew tube, and the protein was extracted and used in the experiment. Protein quantification was performed using Quick Start™ Bradford Reagent (Bio-rad, Hercules, CA, USA). The same amount of protein was subjected to electrophoresis (SDS-PAGE) on SDS-polyacrylamide gel. After transferring the protein separated on the gel through electrophoresis to a polyvinylidene fluoride (PVDF) membrane (Merck Millipore, New Jersey, USA), the product was blocked at room temperature for 1 hour using 5% skim milk/Tris-buffered saline with tween 20 (TBST; 20 mM Tris-HCl, pH 7.4, 150 mM NaCl, 0.1% Tween 20), and reacted with the primary antibody overnight at 4° C. The primary antibodies used herein were anti-PGC1α (1:1000, abcam, ab191838), anti-Akt (1:1000, cell signaling, #9272), anti-phospho Akt (1:1000, cell signaling, #4058), anti-Pi3k (1:1000, cell signaling, #4292), anti-phospho Pi3k (1:1000, cell signaling, #4228), anti-mTOR (1:1000, cell signaling, #2983), anti-MyD88 (1:1000, cell signaling, #4283), anti-Sirt1 (1:1000, cell signaling, #3931), and anti-Traf6 (1:1000, abcam, ab33915), which are diluted in 5% BSA and used. Further, as a loading control, alpha tubulin (1:1000, cell signaling, #2125) was used. For the secondary antibody, horseradish peroxidase (HRP)-conjugated anti-rabbit IgG (1:5000; Cell Signaling, #7074S) was diluted in 5% skim milk and reacted at room temperature for 1 hour. After each reaction, the product was washed three times for 10 minutes with TBST buffer and then proceeded. The washed membrane was visualized using an Enhanced Chemiluminescent (ECL) kit (super-signal west pico plus, 34580, Thermo Scientific, Rockford, IL, USA) that develops bands, and G-box (50S; BI System Co.) was used for quantification and analysis.

### 11. Statistical Analysis

Experimental results were estimated using SPSS package program versuib 25.0 (Statistical Package for the Social Sciences, SPSS Inc., Chicago), one of the computer statistical programs. Student's t-test was conducted to test the significance between the ND group and the HFD group, and between the HFD and CS groups. All results were expressed as mean±S.E (standard error).

### <Experiment Result>

### 1. Analysis of Tissue Weight Change and Body Weight Change by Celery Seed Extract

### 1-1. Variation in Muscle and Organ Weight

In order to analyze changes in muscle and organ weight, gastrocnemius muscle, femoris muscle, tibialis anterior muscle, kidney and liver were extracted and weighed, and then expressed as weight per 100 g of body weight and compared.

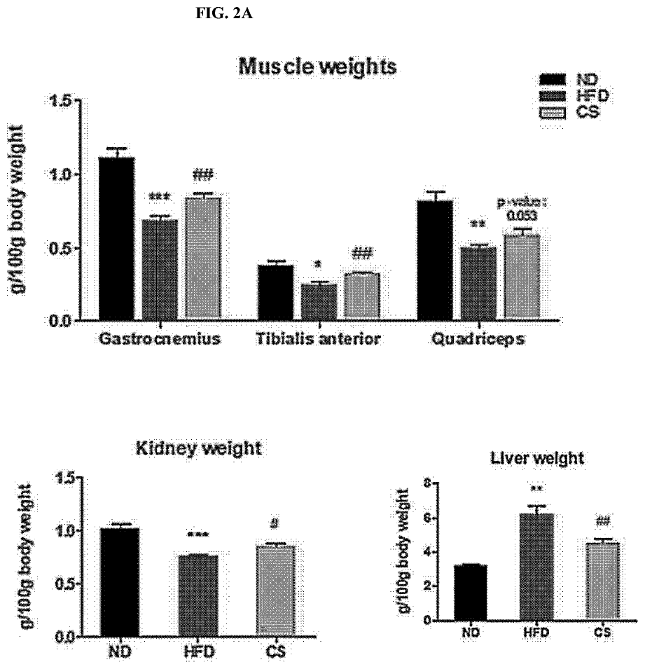

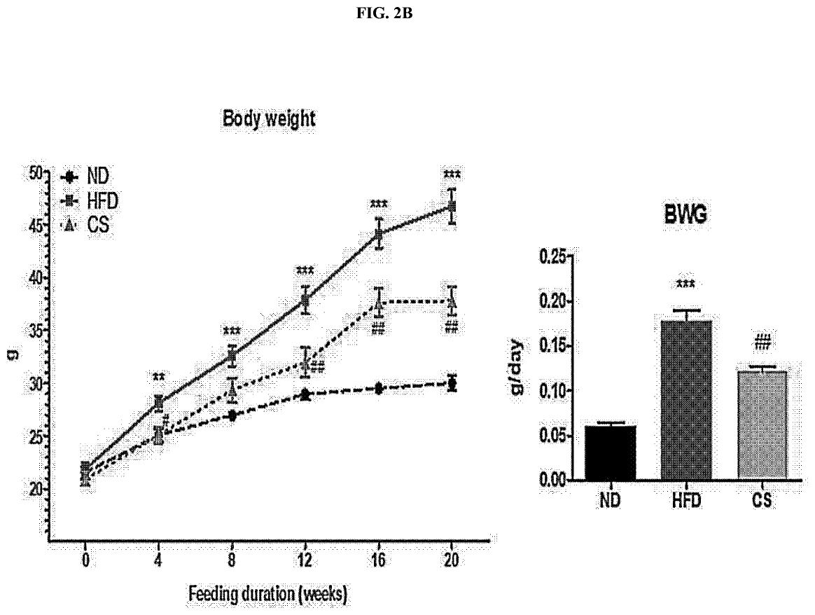

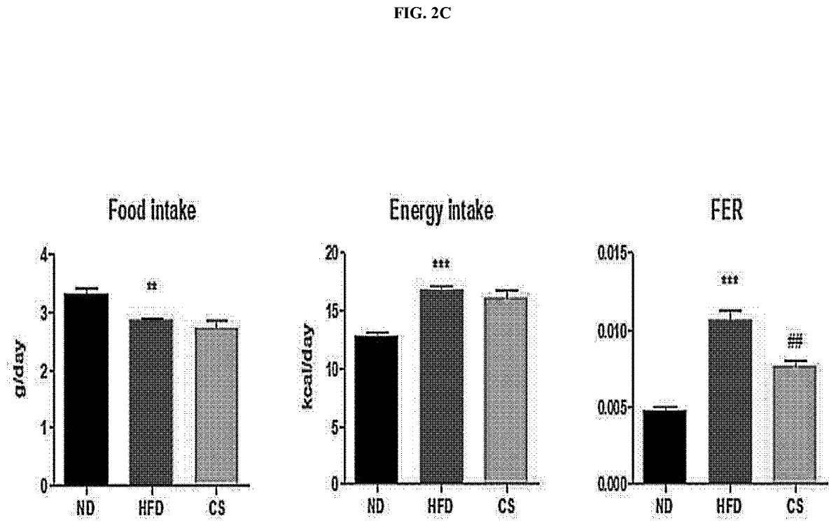

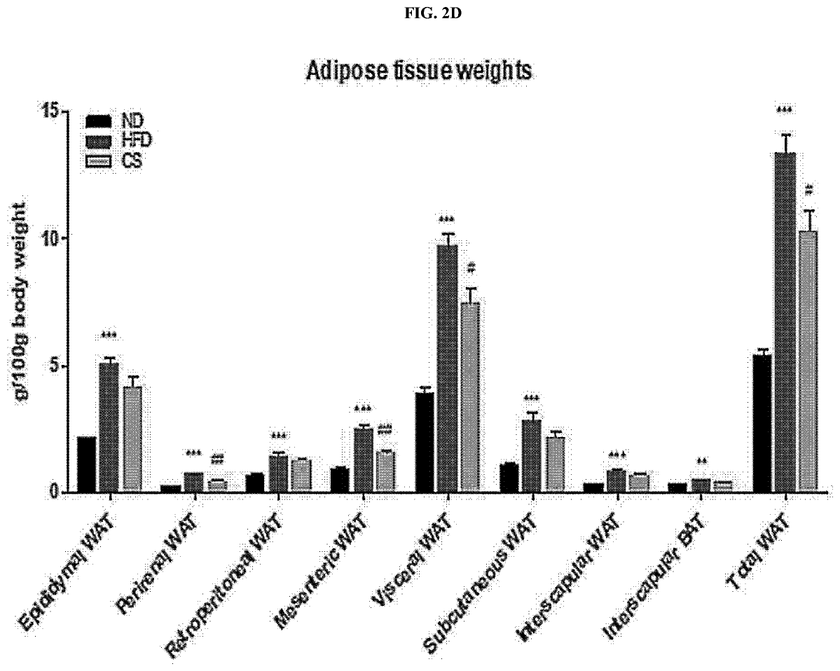

As a result, as shown in FIG. 2a, in the case of muscle, the weight of muscle tissue by all parts of the HFD group was significantly reduced compared to the ND group, while the weight of the gastrocnemius and femoral muscles of the CS group was significantly increased than that of the HFD group. In the case of kidney, the weight of the HFD group decreased significantly compared to the ND group, while the weight of the kidney increased significantly in the CS group compared to the HFD group. In the case of the liver, it was confirmed that the weight of the HFD group was significantly increased compared to the ND group, whereas the weight of the liver was significantly decreased in the CS group than the HFD group. Through the above results, it was confirmed that the celery seed extract had effects of increasing muscle mass, suppressing an increase in liver weight, and suppressing a decrease in kidney weight.

### 1-2. Weight Change

To investigate the effect of intake of celery seed extract on weight gain, body weight and food intake were measured once a week during the breeding period.

As a result, as shown in FIG. 2b, in the case of the HFD group, the body weight increased sharply compared to the ND group, whereas in the case of the CS group, it was confirmed that the body weight significantly decreased from the 12^{th} week with the experimental diet compared the HFD group.

Further, when the body weight gain during the 20 weeks was converted into the weight gain per day (g/day), it was confirmed that the weight gain in the CS group was significantly suppressed compared to the HFD group.

On the other hand, as shown in FIG. 2c, there was no significant difference between the CS group and the HFD group in the daily average food intake and energy intake, but the food efficiency ratio (FER) representing the weight gain to energy intake was found to be significantly increased in the HFD group with high energy density compared to the ND group with low energy density, whereas the CS group showed a significant decrease in the CS group compared to the HFD group. From the above results, it could be seen that the celery seed extract has an effect of significantly reducing body weight in an environment where the same energy is consumed.

### 1-3. Adipose Tissue Weight Change

In order to analyze the change in the weight of the adipose tissue, the adipose tissue of the mouse was excised and weighed, and then expressed as a weight per 100 g of body weight for comparison.

As a result, as shown in FIG. 2d, in the case of the HFD group, since the weight of adipose tissue by all parts significantly increased compared to the ND group, it was confirmed that obesity was induced by the high fat diet. On the other hand, in the CS group supplemented with celery seed extract, the weights of perirenal and mesenteric white adipose tissues corresponding to visceral fat were significantly reduced, and further the weight of visceral fat and total white adipose tissue was also reduced. From the above results, it could be seen that the celery seed extract has effects of reducing body fat by significantly decreasing the weight of white adipose tissue to store energy.

### 2. Analysis of the Effect of Celery Seed Extract on Morphological Changes in Liver Tissue, Adipose Tissue and Muscle Tissue

### 2-1. Muscle Tissue

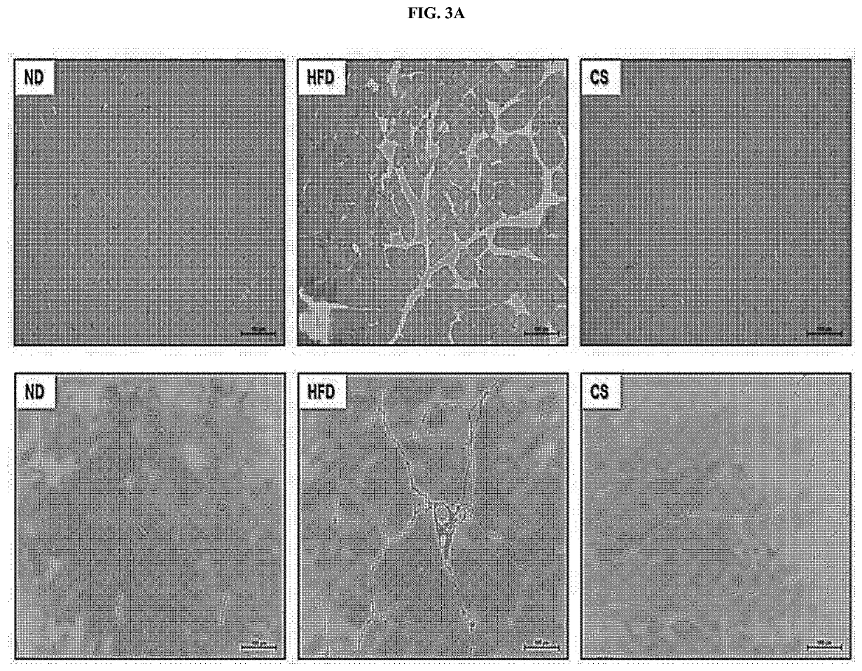

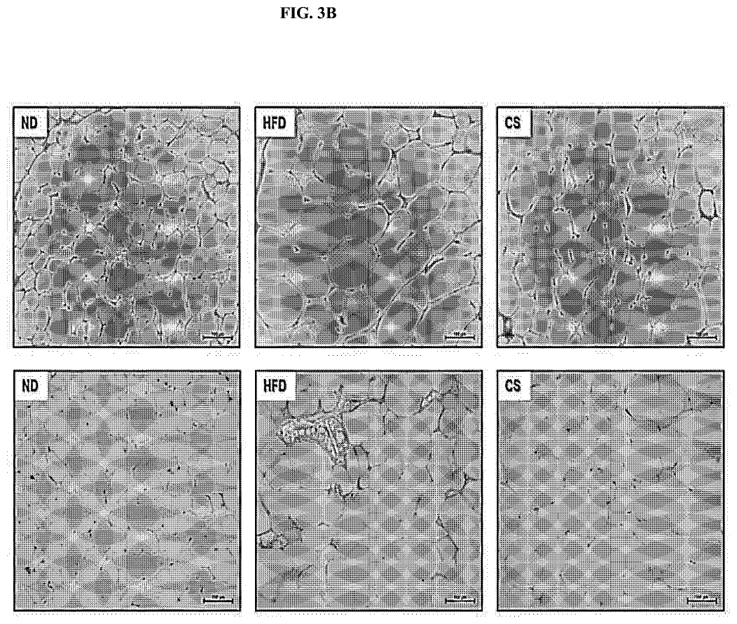

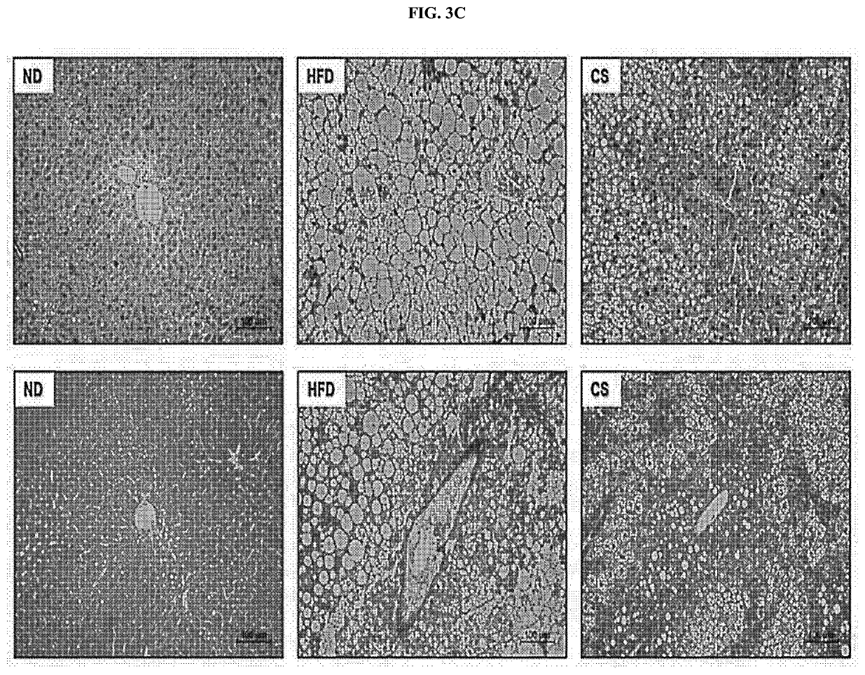

As a result of observing the gastrocnemius muscle of the mouse by H&E staining, as shown in FIG. 3a, the shape and structure of the muscle cell bundle were irregular in the HFD group compared to the ND group, but in the case of the CS group, it could be confirmed that the shape of the muscle cells was improved to a normal level (upper part of FIG. 3a).

Furthermore, as a result of sirius red staining for histological visualization of collagen fibers in muscle tissue, more collagen stained in muscle tissue was observed in the HFD group than in the ND group, but in the case of the CS group, fibrosis of muscle tissue due to high fat diet was shown to be inhibited (lower part of FIG. 3a).

### 2-2. Adipose Tissue

As shown in FIG. 3b, as a result of observing epididymal white adipose tissue (Epididymal WAT) by H&E staining, it was observed that the size of fat cells in the HFD group was larger than that in the ND group, and it was also confirmed that the size of fat cells in the CS group was relatively smaller than that of the HFD group (upper part of FIG. 3b).

Furthermore, as a result of Masson's trichrome staining to examine the effect of celery seed extract on fibrosis of adipose tissue, fibrotic connective tissues were more prominently observed in adipocytes of the HFD group compared to of the ND group. In the case of the CS group, it was found that the fibrosis of adipose tissue due to the high fat diet was inhibited (lower part of FIG. 3b).

### 2-3. Liver Tissue

As a result of H&E staining of the liver tissue, as shown in FIG. 3c, in the case of the HFD group, the largest amount of lipid droplets was accumulated around the portal vein of the liver tissue, but in the case of the CS group supplemented with celery seed extract, the number of fat cells was reduced compared to the HFD group.

Further, in order to investigate effects of the celery seed extract on the fibrosis of liver tissues, Masson's trichrome staining was performed. As a result, in the case of the HFD group, a large amount of connective tissues was accumulated around the portal vein, but in the case of the CS group, it was confirmed that the fibrosis of liver tissue due to the high fat diet tissue was inhibited.

### 3. Analysis of Effect of Celery Seed Extract on Thigh Thickness and Muscle Strength

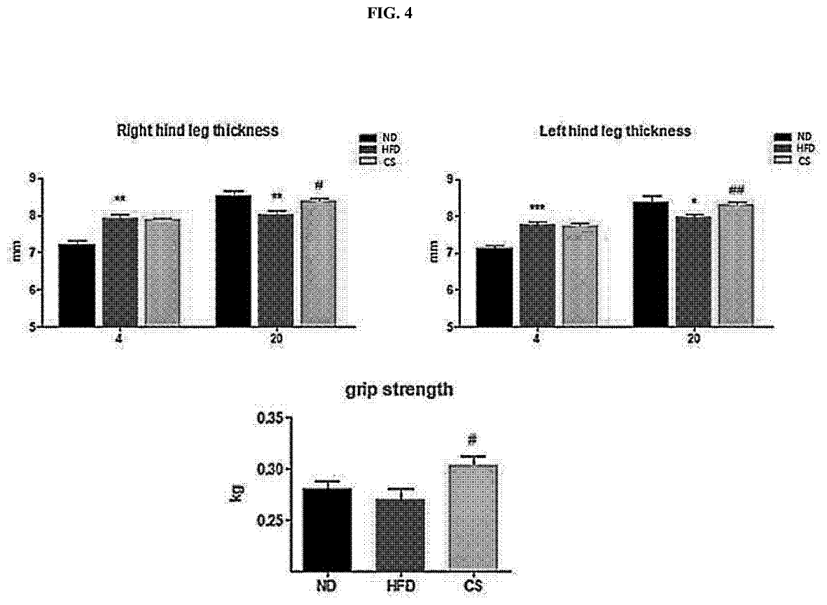

As a result of measuring a thigh thickness of the mouse, as shown in FIG. 4, the thickness of the hind leg thigh measured at the 4th week of feeding the experimental diet was significantly higher in the HFD group than in the ND group, however, no significant difference between the CS group and the HFD group was demonstrated. At the 20th week of feeding the experimental diet, it was confirmed that the thigh thickness of the right and left hind limbs of the CS group increased significantly compared to the HFD group.

Further, as a result of measuring a tensile strength conducted at the 19′ week of the experimental diet, there was no significant difference between the ND group and the HFD group. However, in the case of the CS group, it was confirmed that the tensile strength was significantly increased compared to the HFD group. Therefore, from the above results, it was confirmed that the celery seed extract had effects of improving the thigh thickness reduced by the high fat diet and increasing muscle strength.

### 4. Analysis of the Effect of Celery Seed Extract on Tissue Lipid Content

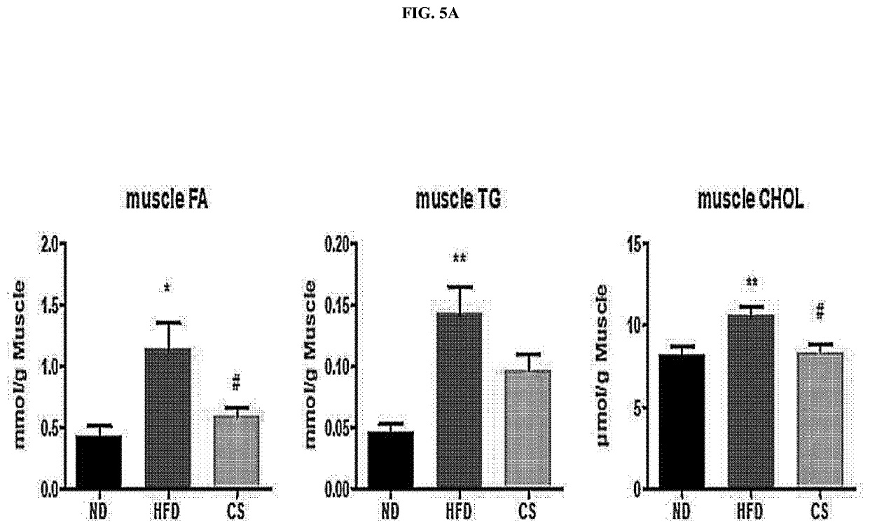

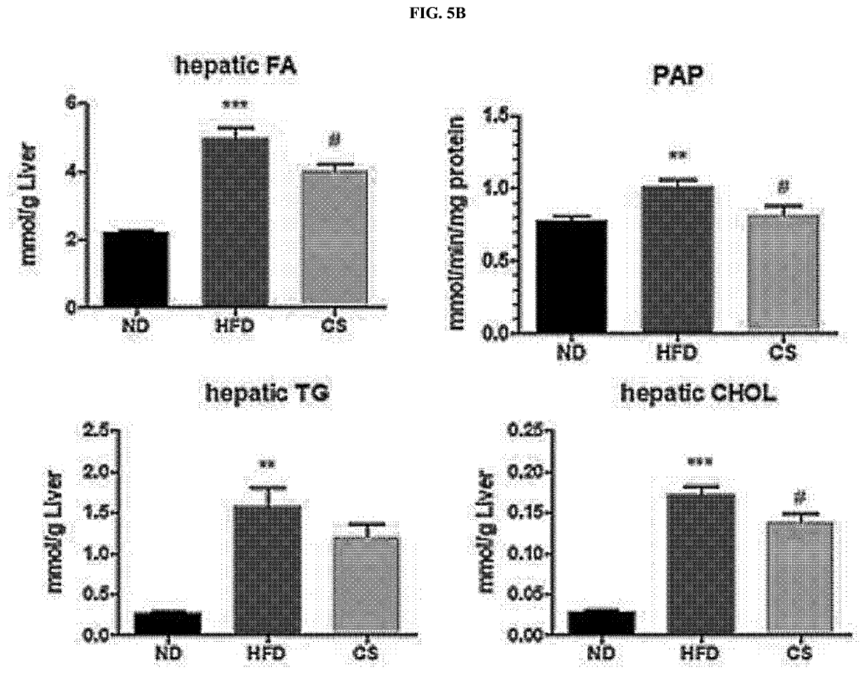

In order to analyze the effects of the celery seed extract on the lipid content of tissue, the results of comparing free fatty acid, triglyceride and cholesterol contents per unit weight of tissue are shown in FIG. 5.

### 4-1. Lipid Content of Muscle Tissue

As shown in FIG. 5a, it was confirmed that the triglyceride, free fatty acid and cholesterol contents of the muscle tissue were significantly increased in the HFD group, whereas the free fatty acid and cholesterol contents in the CS group were significantly decreased compared to the HFD group. From the above results, since the celery seed extract suppresses the increase in lipid content of muscle tissue, the above sections 1-1 and 3, showed that the increased muscle weight and thickness were not due to lipid accumulation.

### 4-2. Lipid Content of Liver Tissue and Activity of Lipid Metabolism Enzymes

As a result of measuring the activity of phosphatidate phosphohydrolase (PAP), an enzyme related to triglyceride synthesis in liver tissue, as shown in FIG. 5b, the HFD group was observed to be significantly higher than the ND group, and the increased PAP activity due to the high fat diet was found to be significantly reduced by supplementing with celery seed extract.

Further, in the case of the HFD group consuming the high fat diet, the lipid content of liver tissue increased significantly compared to the ND group consuming a normal diet, but in the case of the CS group supplemented with celery seed extract, it was confirmed that free fatty acids in liver tissue and total cholesterol content were significantly reduced compared to the HFD group. From the above results, it was confirmed that the celery seed extract reduced the lipid content of liver tissue.

### 5. Analysis of Effect of Celery Seed Extract on the Expression of Proteins Related to Muscle Cell Growth

To confirm the effect of celery seed extract on the cell growth mechanism of muscle tissue, the protein expression level of the Pi3k/Akt/mTOR pathway, which promotes muscle cell differentiation and is involved in muscle fiber protein accumulation, was measured.

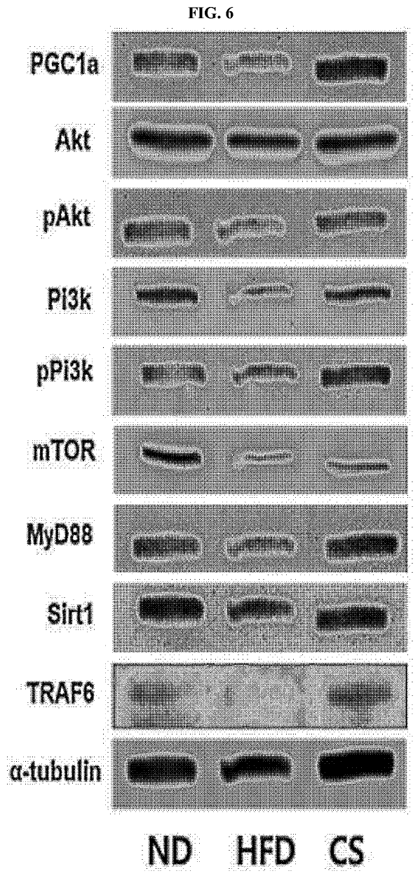

As a result, as shown in FIG. 6, PGC1α (peroxisome proliferator-activated receptor gamma coactivator 1-alpha) protein involved in inhibiting inflammation and muscle atrophy in muscle tissue, and MyD88 (Myeloid differentiation primary response 88) as a parent factor of the Pi3k/Akt/mTOR pathway and Traf6 (Tumor necrosis factor receptor associated factor 6) proteins showed a significant increase in expression in the CS group compared to the HFD group.

On the other hand, in the case of Akt (Protein kinase B) and Pi3k (phosphoinositide 3-kinase) proteins involved in myocyte differentiation and apoptosis inhibition, it was confirmed that the expression of pAkt and pPi3k proteins, which are activated forms of Akt and Pi3k, increased. From the above results, it was confirmed that the celery seed extract has an effect of improving muscle metabolism.

### 6. Analysis of Effect of Celery Seed Extract on Plasma Lipid Concentration

### 6-1. Plasma Triglyceride and Total Cholesterol Concentrations

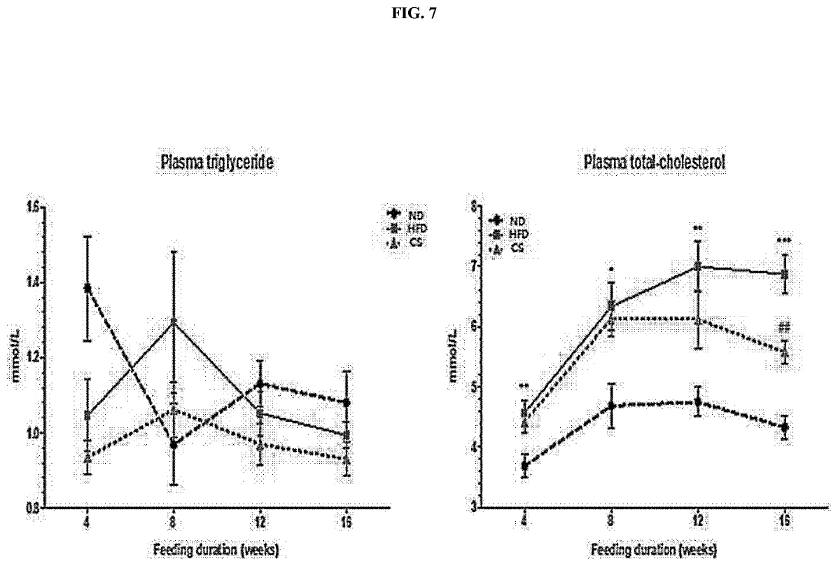

As a result of measuring plasma triglyceride and total cholesterol concentrations once a week during the experimental period, the total cholesterol concentrations of the HFD group increased significantly compared to the ND group after 4 weeks of feeding the experimental diet, as shown in FIG. 7. In the case of the CS group, it was confirmed that plasma total cholesterol concentration was significantly reduced compared to the HFD group at 16 weeks of feeding the experimental diet.

On the other hand, triglyceride concentration did not show a significant difference between groups.

### 6-2. Plasma Lipid Concentration

Free fatty acids, triglycerides, total cholesterol concentrations, and non HDL cholesterol concentrations in plasma were significantly decreased in the CS group compared to the HFD group, whereas the ratio of HDL cholesterol to total cholesterol (HDL-C/Total C ratio; HTR) was significantly increased in the CS group compared to the HFD group. Furthermore, it was confirmed that the levels of atherogenic intex (AI) and plasma apolipoprotein B (Apo B) were significantly lower in the CS group than in the HFD group. Therefore, it was found from the above results that the celery seed extract reduced the plasma lipid concentration.

TABLE 2

| | Experimental groups | | |
|---|---|---|---|
| Measurements | ND | HFD | CS |
| Free fatty acids | 1.14 ± 0.04 | 1.08 ± 0.02 | 0.99 ± 0.02 |
| (mol/L) | | | |
| Triglycerides | 1.02 ± 0.12 | 1.00 ± 0.06 | 0.80 ± 0.04 ^{#} |
| (mmol/L) | | | |
| Total cholesterol | 3.86 ± 0.22 | 7.61 ± 0.56 ** | 5.27 ± 0.14 ^{##} |
| (mmol/L) | | | |
| HDL-cholesterol | 0.94 ± 0.06 | 1.68 ± 0.05 *** | 1.45 ± 0.09 |
| (mmol/L) | | | |
| nonHDL- | 2.92 ± 0.17 | 5.93 ± 0.52 | 3.82 ± 0.08 ^{#} |
| cholesterol | | | |
| (mmol/L) | | | |
| HTR (%) | 24.47 ± 0.84 | 22.88 ± 1.36 | 27.48 ± 1.23 ^{#} |
| AI | 3.11 ± 0.14 | 3.45 ± 0.23 | 2.69 ± 0.18 ^{#} |
| Apo A-1 (mg/ml) | 0.29 ± 0.02 | 0.35 ± 0.03 | 0.37 ± 0.03 |
| Apo B (mg/ml) | 0.22 ± 0.01 | 0.40 ± 0.05 * | 0.24 ± 0.02 ^{#} |
| Apo B/Apo A-1 | 0.78 ± 0.07 | 1.13 ± 0.11 * | 0.66 ± 0.05 ^{##} |

### 7. Analysis of Effect of Celery Seed Extract on Plasma Hepatotoxicity Index

In order to confirm the effect of celery seed extract on hepatotoxicity, the activities of glutamic oxaloacetic transaminase (GOT) and glutamic pyruvic transaminase (GPT), which are closely related to liver damage, were analyzed using an enzyme kit from Asan Pharm Co., Seoul, Korea.

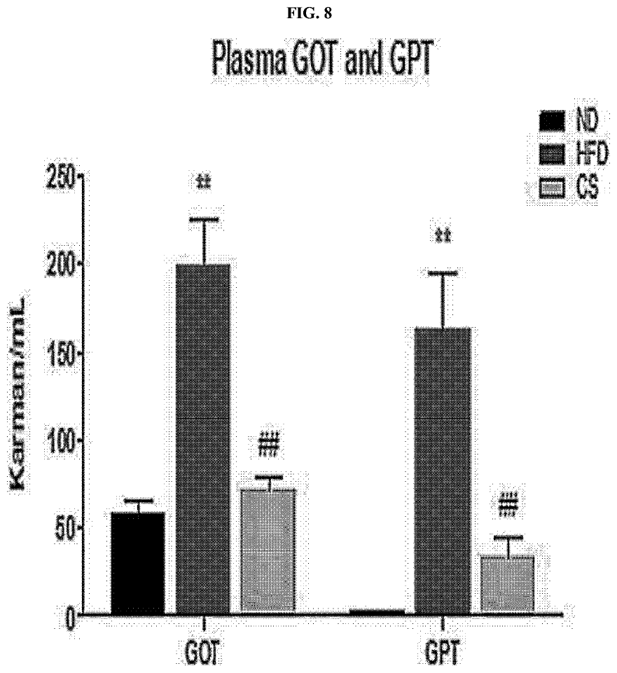

As a result, as shown in FIG. 8, in the case of the HFD group, plasma GOT and GPT levels were significantly increased compared to the ND group, and in the case of the CS group, plasma GOT and GPT levels were significantly decreased compared to the HFP group. From the above results, it was confirmed that the celery seed extract has effects of inhibiting hepatotoxicity.

### 8. Analysis of Effect of Celery Seed Extract on Antioxidant-Related Biomarkers

### 8-1. Paraoxonase (PON) Activity

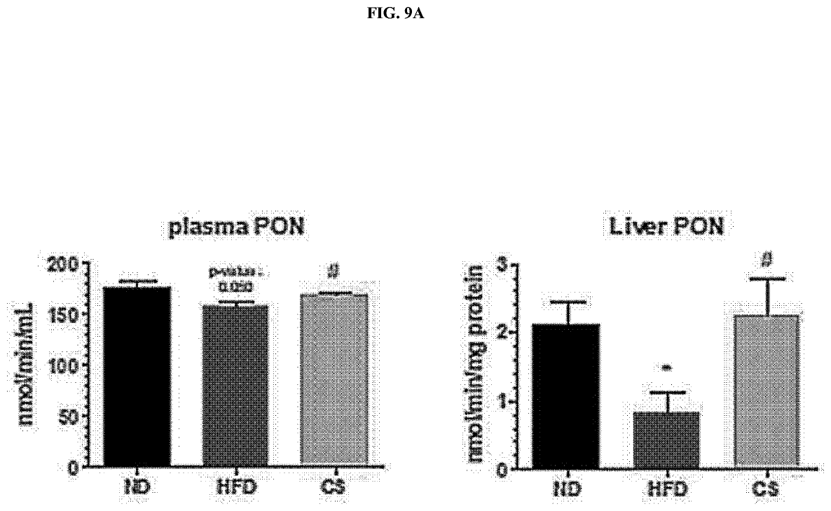

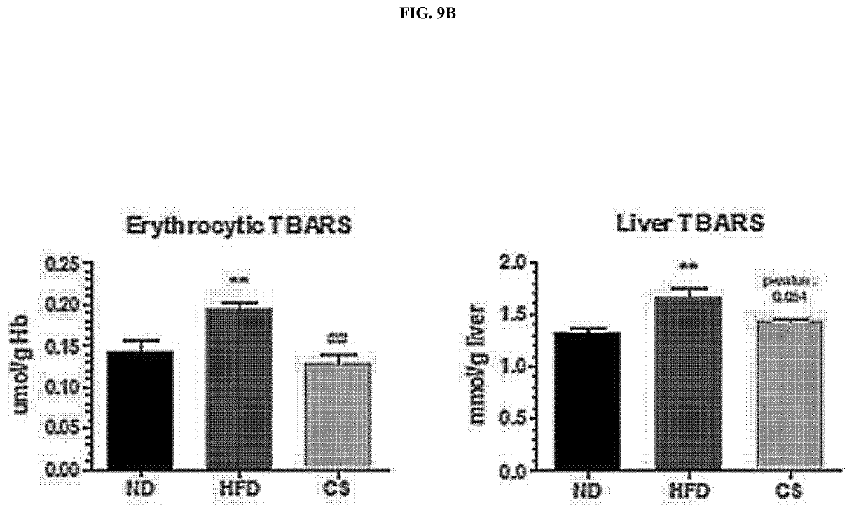

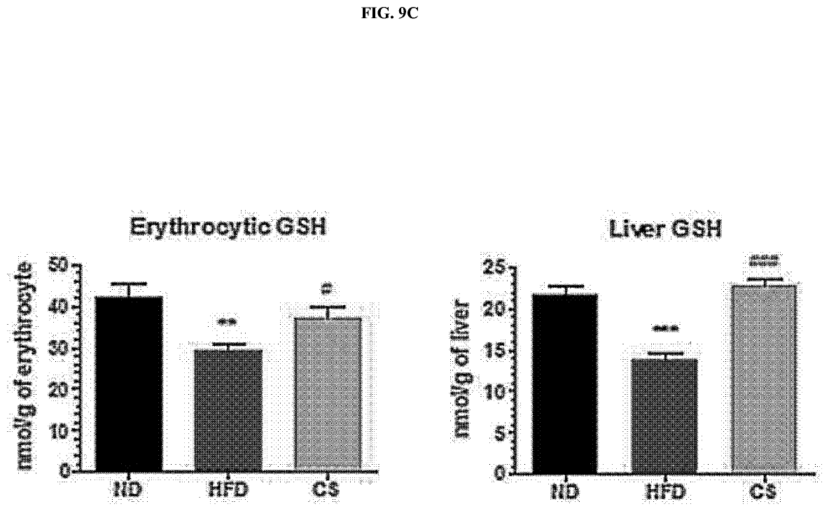

As a result of comparison of PON activity that inhibits oxidation of plasma lipoprotein particles, as shown in FIG. 9a, in the case of plasma PON activity, the HFD group showed a tendency to decrease compared to the ND group, whereas it was found that the CS group showed significant increase compared to the HFD group. Further, in the case of liver tissue PON activity, the HFD group showed the lowest activity, and it was confirmed that the CS group increased significantly compared to the HFD group.

### 8-2. Lipid Peroxides (Thiobarbituricacid Reactive Substance; TBARS)

As a result of comparison of TBARS of red blood cells and liver tissue, as shown in FIG. 9b, in the case of red blood cell TBARS, the HFD group significantly increased compared to the ND group, but the CS group significantly decreased compared to the HFD group. Further, in the case of liver tissue TBARS, it was confirmed that the HFD group increased significantly compared to the ND group, but the CS group showed a decreasing tendency.

### 8-3. Glutathione (GSH)

As a result of comparing the content of GSH, an antioxidant-related biomarker, in red blood cells and liver tissue, as shown in FIG. 9c, it was confirmed that the content increased compared to the HFD group. From the above results, it was confirmed that the celery seed extract has effects of suppressing the increase in lipid peroxides and increasing the antioxidant activity.

### Example II. Effect of Celery Seed on Geriatric Muscle Loss

### Experiment Materials and Methods

### 1. Preparation of Celery Seed Extract

Celery see powder was purchased from ES Food Ingredients Co., Ltd. and used. After adding 1 L of 70% ethanol to 100 g of celery seed powder as a raw material, extraction was repeated three times for 3 hours at 60° C., and the extracted solution was filtered, concentrated under reduced pressure, and lyophilized. The celery seed spirit extract powder was obtained and stored frozen and used for dietary preparation, and the yield of the extract was 10.02%.

### 2. Experimental Animal Models

### 2-1. Laboratory Animal

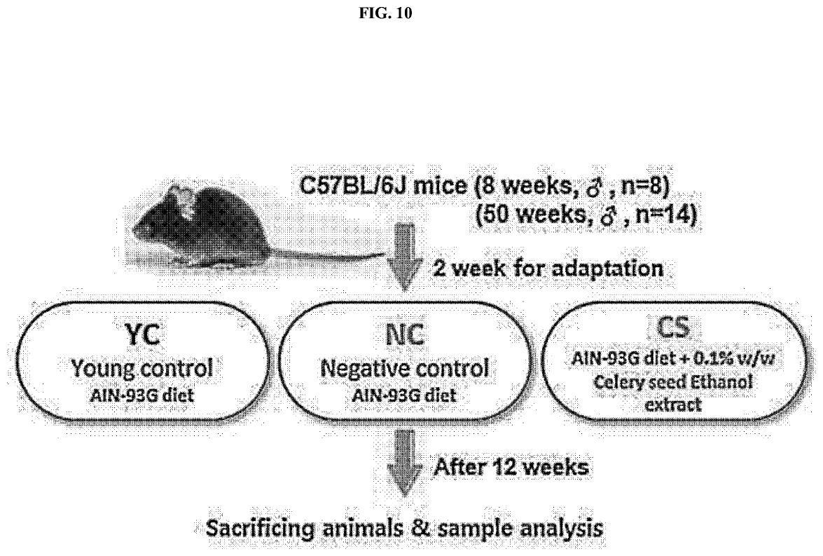

As experimental animals, 8-week-old male C57BL/6J mice and 50-week-old male C57BL/6J mice (JA BIO, Korea) were purchased from JoongA Bio and used. As shown in FIG. 10, after adaptation by feeding a diet in the form of pellets for 2 weeks, the experimental animals were divided into a young mouse group (YC, Young control, n=8), an aged mouse group (NC, Negative control, n=7), and an aged mouse group provided with a diet supplemented with celery seed extract (CS, 0.1% (w/w) celery seed ethanol extract, n=7), followed by feeding with an experimental diet for 12 weeks. In the animal breeding room, constant conditions were maintained with a predetermined temperature (24±2° C.), predetermined humidity (50±5%), and a photoperiod of 12 hours apart (AM 6:00˜PM 18:00). and drinking water were provided ad libitum. During the 12-weeks experimental period, food intake and body weight were measured once a week, and a value obtained by dividing daily weight gain by daily energy intake was used as food efficiency ratio (FER).

### 2-2. Composition of the Experimental Diet

The composition of the experimental diet is as shown in Table 3. AlN-93G diet was prepared and fed to all groups, and the CS group was fed by adding celery seed extract at a dose of 0.10% of the diet to the normal diet.

TABLE 3

| | | | CS |
|---|---|---|---|
| | YC | NC | (AIN-93G |
| | (AIN-93G | (AIN-93G | diet + celery |
| Ingredient (g) | diet) | diet) | seed 0.1%) |
| Casein (from milk) | 200.0 | 200.0 | 200.0 |
| Corn Starch | 397.5 | 397.5 | 397.5 |
| Sucrose | 100.0 | 100.0 | 100.0 |
| Maltodextrin | 132.0 | 132.0 | 132.0 |
| Cellulose | 50.0 | 50.0 | 50.0 |
| Soybean Oil | 70.0 | 70.0 | 70.0 |
| Mineral Mix, AIN- | 35.0 | 35.0 | 35.0 |
| 93G-MX | | | |
| Vitamin Mix, AIN- | 10.0 | 10.0 | 10.0 |
| 93-VX | | | |
| TBHQ, antioxidants | 0.014 | 0.014 | 0.014 |
| L-Cystine | 3.0 | 3.0 | 3.0 |
| Choline bitartrate | 2.5 | 2.5 | 2.5 |
| Celery Seed | | | 1.00 |
| Extract | | | |
| Total (g) | 1000.01 | 1000.04 | 1001.04 |

### 3. Sample Collection

At the end of breeding, the experimental animals were fasted for 12 hours, anesthetized using isoflurane (5 mg/kg body weight, Baxter, USA), and blood was collected from the inferior vena cava. The collected blood was treated with heparin, centrifuged at 3,000 rpm and 4° C. for 15 minutes, and plasma was collected and stored at −70° C. until sample analysis.

Liver, kidney, epididymal white fat, perirenal white fat, interscapular white/brown fat, and muscle tissue were excised from the experimental animals, rinsed several times in a saline solution (0.9% saline solution) and, after removing water on the surface, weighed. The treated part was divided according to the purpose of the experiment, and rapidly cooled in liquid nitrogen and stored at 70° C. until analysis.

### 4. Measurement of Thigh Thickness and Tensile Strength

During the experiment period, the thigh thickness of a mouse was measured at 4-week intervals using a caliper.

In order to determine the tensile strength of the experimental animals at the 12th week of feeding the experimental diet, the tensile strength was measured continuously for 5 days after adaptation in a measuring device for 3 days.

Specifically, the experimental animal was placed on top of a grid of the measuring device and allowed it to grasp the grid with all four feet so that, when holding the mouse's body and tail while keeping the same in parallel to the grid, and then, gently and slowly pulling the tail backward, a maximum muscular strength by the mouse forcibly gripping the grid was recorded.

### 5. Biochemical Analysis

### 5-1. Plasma Lipid Concentration Analysis

Plasma triglyceride, total cholesterol, and HDL-cholesterol concentrations were measured using an enzyme kit from Asan Pharm Co., Seoul, Korea. Free fatty acid content was measured using a test solution for measuring free fatty acid (Non-Esterified fatty acid, NEFA kit, Shinyang Chemical) using the principle of a coloring method using an enzymatic method.

### 5-2. Tissue Lipid Content Measurement

Lipid content of liver and muscle tissue was extracted according to the method of Folch et al. (1957), and then the extract was volatilized with nitrogen gas at 37° C. and diluted with isopropanol. For quantification, the enzyme reagent was mixed with 3 mM cholic acid as an emulsifier and 0.5% Triton X-100 to remove turbidity during color development.

### 5-3. Enzyme Source Separation

5-3-a. Erythrocyte Enzyme Source Isolation

Erythrocytes were centrifuged at 3,000 rpm and 4° C. for 15 minutes according to the method of McCord and Fridovich (1969) to completely remove plasma and buffy coat, and then washed three times with 0.9% physiological saline. The washed red blood cells were lysed with the same amount of distilled water and used for the measurement of antioxidant enzyme activity.

5-3-b. Liver Tissue Enzyme Source Isolation

For the isolation of enzyme sources in liver tissue, the isolation method conducted by Hulcher et al. (1973) was partially modified and applied. 0.5 g of liver tissue was homogenized with a glass teflon homogenizer (Glascol, 099C K44, USA) in an ice-cooled state by adding a buffer solution containing 0.1 M triethanolamine, 0.02 M ethylenediamine tetracetate (EDTA, pH 7.4), and 0.002 M dithiothreitol (DTT). After that, centrifugation was performed at 3,000 rpm and 4° C. for 15 minutes, and only the supernatant was centrifuged again at 13,000 rpm and 4° C. for 15 minutes. Among them, the precipitate in a lower layer separated from the supernatant was used as the mitochondrial fraction, and the supernatant was treated for 1 hour at 32,500 rpm and 4° C. using an ultracentrifuge (Beckman, Optima TLX-120, USA) to thus obtain cytosol fraction. For the microsomal fraction, the same buffer solution was added to the isolated pellets as well as the cytoplasmic fraction of the supernatant, followed by ultracentrifugation again at 33,000 rpm and 4° C. for 40 minutes, and then the pellets were dissolved in 1 ml buffer solution and stored at −70° C. for further analysis and protein quantification.

### 5-4. Measurement of Antioxidant Enzyme Activity

5-4-a. Determination of Glutathione Content

Glutathione (GSH) content includes both oxidized GSH and reduced GSH, and was measured by modifying and supplementing the method of Fiala et al. (1976). 0.5 g of liver tissue was added to a buffer solution containing 0.1 M triethanolamine, 0.02 M EDTA (pH 7.4) and 0.002 M DTT, and homogenized with a glass teflon homogenizer (Glascol, 099C K44, USA) in an ice-cooled state. 0.3 mL of distilled water and 0.5 mL of 4% sulfosalicylic acid were added to 0.2 mL of the homogenate, followed by centrifugation at 25,000 rpm and 4° C. for 10 minutes to obtain a supernatant. 2.7 mL of 0.1 M disulfide reagent (5.5′-dithiobis+0.1 M sodium phosphate buffer, pH 8.0) was added to 0.3 mL of the supernatant, reacted for 20 minutes at room temperature, and absorbance was measured at 412 nm.

5-4-b. Measurement of Superoxide Dismutase (SOD) Activity

SOD is an enzyme that catalyzes the decomposition of superoxide anion radical (O²—) into H₂O₂ and O₂. The activity measurement was performed by modifying and supplementing the method of Marklund et al. (1974) and using the degree of color development by autooxidation of pyrogallol in an alkaline state. 0.1 mL of liver tissue and red blood cell enzyme source and 0.1 mL of 7.2 mM pyrogallol solution were added to 1.5 mL of 50 mM Tris-hydroxymethyl-aminomethane buffer (pH 8.5) containing 10 mM EDTA, reacted at 25° C. for 10 minutes, followed by adding 50 μL of 1 N HCl solution to thus terminate the reaction. Further, a change in absorbance was measured at 420 nm (Beckman 650 spectrophotometer, USA) to calculate the same in terms of SOD unit to cytosolic protein and red blood cell hemoglobin required to prevent autoxidation of pyrogallol (SOD unit/mg cytopsolic protein, SOD unit/g hemoglobin).

### 6. Intraperitoneal Glucose Tolerance Test

After the mice were fasted for 12 hours, a glucose solution was intraperitoneally administered at 0.5 g per kg body weight, and blood was collected through the tail vein after 0, 30, 60, and 120 minutes, respectively, and measured using a glucometer.

### 7. Liver Tissue Toxicity Analysis by Measuring Plasma GOT and GPT Activity

GOT and GPT activities, which are closely related to hepatocellular damage, were measured using an enzyme kit from Asan Pharm Co., Seoul, Korea.

### 8. Morphological Analysis of Tissue Cells

For morphological observation of the tissue, a part of the muscle tissue excised at the time of animal sacrifice was fixed in 10% formaldehyde solution for 24 hours, exchanged twice with the same solution, dehydrated with 2-fold ethanol and embedded in paraffin. Then, poly-tissue sections having a thickness of 5 μm treated with L-lysine were prepared, stained with H&E, and observed under an optical microscope at 200× magnification. To stain muscle fibers, sirius red staining was performed and observed under an optical microscope at 200× magnification. For immunochemical analysis, Igf-1R and Myostatin were stained by Immunohistochemistry (IHC) staining and observed under a light microscope at 400× magnification.

### 9. Real-Time PCR

RNA was isolated from muscle tissue and cDNA was synthesized using the Prime Script Real time reagent kit (Takara, Japan). cDNA was diluted in RNAse free water, stored at −20° C., and used when performing real-time RT-PCR. For real-time RT-PCR gene expression analysis, SYBR Green PCR kit (Takara, Japan) was used. Primers capable of analyzing the expression of each gene were synthesized by Genotech Co., Ltd. (Daejeon, Korea). At this time, the threshold cycle (Ct) was analyzed by monitoring the fluorescence signal for each cycle, and the mRNA expression between the experimental groups was quantitatively analyzed with the CFX96 real time system (Bio-rad, USA). The primers used herein are shown in Table 4.

TABLE 4

| | Direction | |
|---|---|---|
| Primer | of primer | Sequence (5′→3′) |
| Atrogin | Forward | 5′-AACCGGGAGGCCAGCTAAAGAACA-3′ |
| | | (SEQ ID NO: 1) |
| | Reverse | 5′-TGGGCCTACAGAACAGACAGTGC-3′ |
| | | (SEQ ID NO: 2) |
| MuRF1 | Forward | 5′-GAGAACCTGGAGAAGCAGCT-3′ |
| (Muscle RING-Finger | | (SEQ ID NO: 3) |
| protein 1) | Reverse | 5′-CCGCGGTTGGTCCAGTAG-3′ |
| | | (SEQ ID NO: 4) |
| FoxO1 | Forward | 5′-GTACGCCGACCTCATCACCA-3′ |
| (Forkhead Box O1) | | (SEQ ID NO: 5) |
| | Reverse | 5′-TGCTGTCGCCCTTATCCTTG-3′ |
| | | (SEQ ID NO: 6) |
| FoxO3 | Forward | 5′-TCGCCTCCTGGCGGGCTTA-3′ |
| (Forkhead Box O3) | | (SEQ ID NO: 7) |
| | Reverse | 5′-ACGGCGGTGCTAGCCTGAGA-3′ |
| | | (SEQ ID NO: 8) |

### 10. Western Blot Measurement

0.1 g of frozen muscle tissue was homogenized by adding 3 mm beads and 1 ml of lysis buffer (T-PER buffer, Thermo Scientific, Rockford, IL, USA), centrifuged at 14,000 rpm for 15 minutes, and only the supernatant was collected and transferred to a new tube, followed by extracting and using the protein in the experiment. Protein quantification was performed using Quick Start™ Bradford Reagent (Bio-rad, Hercules, CA, USA). The same amount of protein was subjected to electrophoresis (SDS-PAGE) on SDS-polyacrylamide gel. After transferring the protein divided on the gel through electrophoresis to a polyvinylidene fluoride (PVDF) membrane (Merck Millipore, New Jersey, USA), blocking was proceeded using 5% skim milk/Tris-buffered saline with tween 20 (TBST; 20 mM Tris-HCl, pH 7.4, 150 mM NaCl, 0.1% Tween 20) for 1 hour at room temperature and a reaction with primary antibody was performed overnight at 4° C. A secondary antibody was diluted in 5% skim milk of horseradish peroxidase (HRP)-conjugated anti-rabbit IgG (1:5000; Cell Signaling, #7074S) and reacted at room temperature for 1 hour. After each reaction, it was washed three times for 10 minutes with TBST buffer and then further proceeded. The washed membrane was visualized using an Enhanced Chemiluminescent (ECL) kit (super-signal west pico plus, 34580, Thermo Scientific, Rockford, IL, USA) that develops bands, and quantification and analysis were performed using G-box (50S; BI System Co.)

### 11. Analysis of Short-Chain Fatty Acids

A certain amount of the obtained biofeces was weighed, diluted with tertiary distilled water according to the ratio, sufficiently stirred, and then centrifuged at 13,000 rpm for 3 min at room temperature (RT).

After transferring 150 μL of the supernatant to a vial for short-chain fatty acid analysis, 150 μL of GC buffer was dispensed. It was sealed with a short-chain fatty acid analysis cap and analyzed for 10 minutes using an Agilent Headspace 7697A+Agilent GC 7890 B+Flame lpnization Detector equipped with an HP-lnnowax column. The short-chain fatty acids to be analyzed were prepared and mixed at the optimum concentration for analysis, followed by serial dilution with tertiary distilled water, and analysis was repeated 5 times for each concentration, starting with a low concentration, to derive the results.

### 12. Intestinal Microorganism Analysis Method

For biofecal bacterial DNA extraction, Mag-Bind® Universal Pathogen Kit (Omega Bio-tek) was used. According to the manufacturer's protocol, fecal samples were suspended in SLX-Mlus Buffer, pulverized, separated and washed. The extracted fecal microbial DNA was amplified with 16S Amplicon PCR Forward Primer (5′-TCGTCGGCAGCGTCAGATGTATAAGAGACAGCCTACGGGNGGCWGCAG-3′; SEQ ID NO:9) and 16S Amplicon PCR Reverse Primer (5′-GTCTCGTGGGCTCGGAGATGTGTATAAGACCAGAGTATCACTACH; SEQ ID NO:10). PCR was performed at 95° C. for 3 minutes, and it was maintained at 4° C. for 8 cycles of PCR reaction, each including: 95° C. for 30 seconds: 55° C. for 30 seconds; 72° C. for 30 seconds; and 72° C. for 5 minutes, respectively. After the cleanup step, a concentration of the library was checked using Qubit 4.0 (ThermoFisher Scientific) along with 1×dsDNA HS Assay Solution (ThermoFisher Scientific), and then, sequenced using the Illumina Miseq system. Reads were aligned using a unique barcode for each PCR product. Sequencing results were analyzed using the Qiime2 bioinformatics pipeline, and taxonomic assignments were performed with the Silva reference database.

### 13. Statistical Analysis

Experimental results were calculated using SPSS package program version 25.0 (Statistical Package for the Social Sciences, SPSS Inc., Chicago), one of the computer statistical programs. Student's t-test was conducted to test the significance between each YC group and the NC group and between the NC group and each CS group. All results were expressed as mean±S.E (standard error).

### <Experiment Result>

### 1. Weight Change by Celery Seed Extract

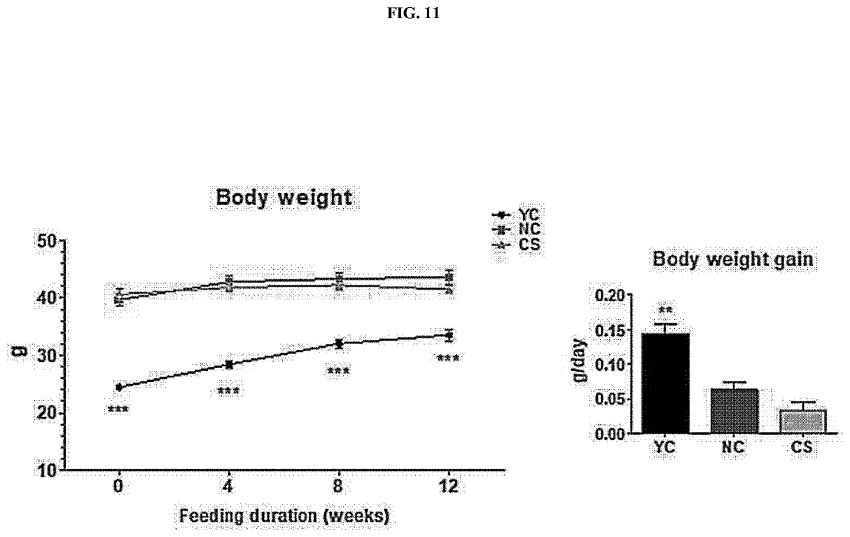

The average weight change during 12 weeks of feeding the experimental diet is shown in FIG. 11. There was a significant difference in body weight between the YC and NC groups from before the start of the experiment to the end of the experiment, but there was no significant difference in body weight and weight gain between the NC and CS groups.

### 2. Effect of Celery Seed Extract on Dietary Intake and Dietary Efficiency

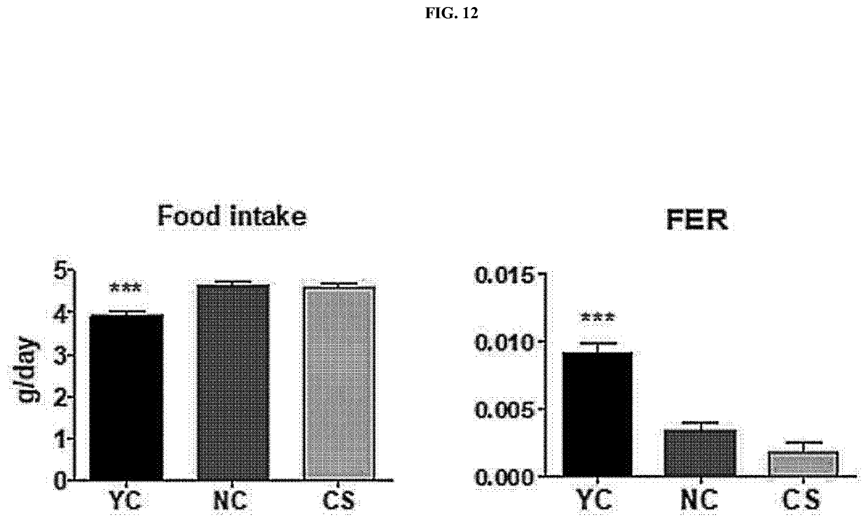

As shown in FIG. 12, the average food intake per day (g/day) increased in the NC group compared to the YC group, but there was no significant difference between the CS and NC groups. Food efficiency ratio (FER) increased significantly in the YC group, where weight increased due to growth, compared to the NC group, which had little weight change. On the other hand, there was no significant difference between the NC and CS groups.

### 3. Effect of Celery Seed Extract on Adipose Tissue Weight

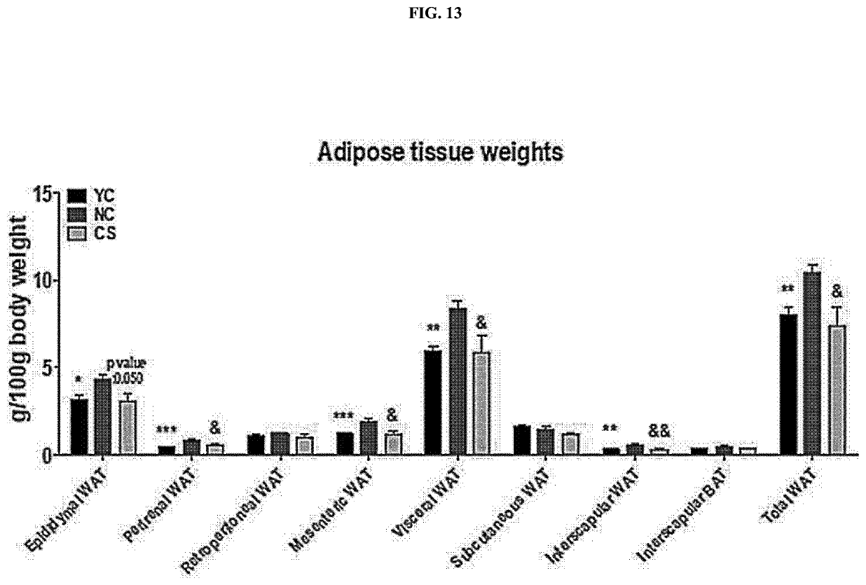

As a result of measuring adipose tissue weight per unit body weight, as shown in FIG. 13, the weights of epididymal white adipose tissue, perirenal white adipose tissue, mesenteric white adipose tissue, visceral white adipose tissue, interscapular white adipose tissue, and total white adipose tissue of the NC group significantly increased compared to the YC group. Further, with the supplementation of celery seed extract, it was observed that the weights of epididymal white adipose tissue, perirenal white adipose tissue, mesenteric white adipose tissue, visceral adipose tissue, interscapular white adipose tissue, and total white adipose tissue were significantly reduced.

### 4. Effects of Celery Seed Extract on Muscle Tissue Weight, Tensile Strength and Hind Thigh Thickness

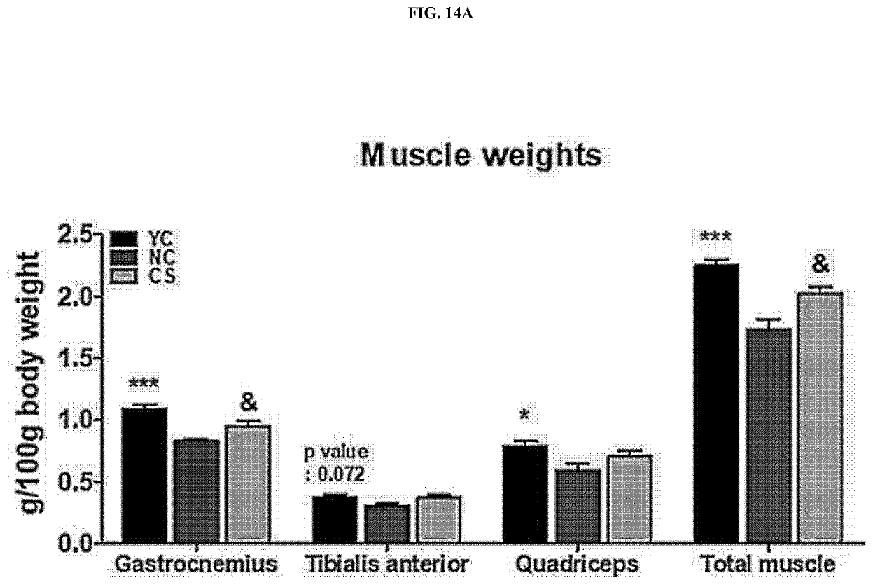

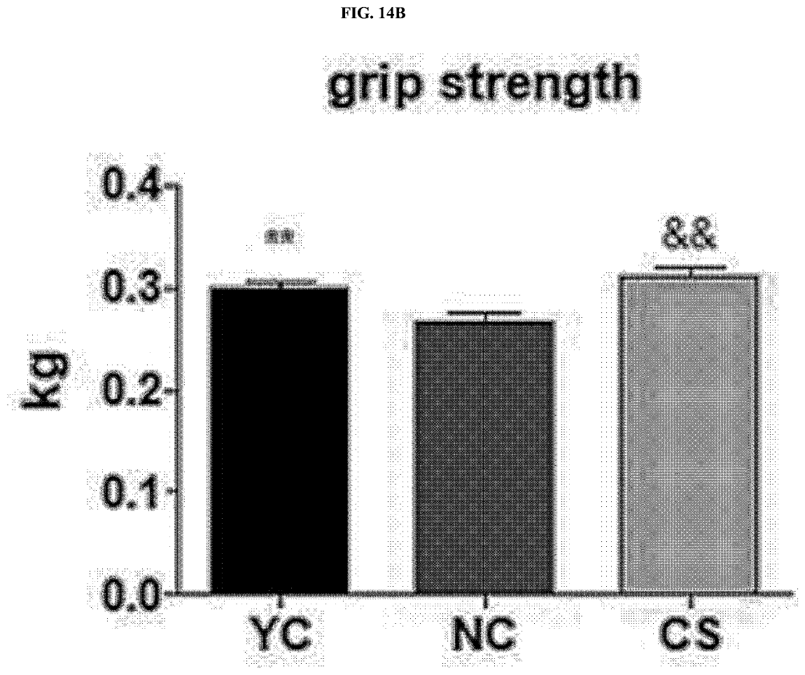

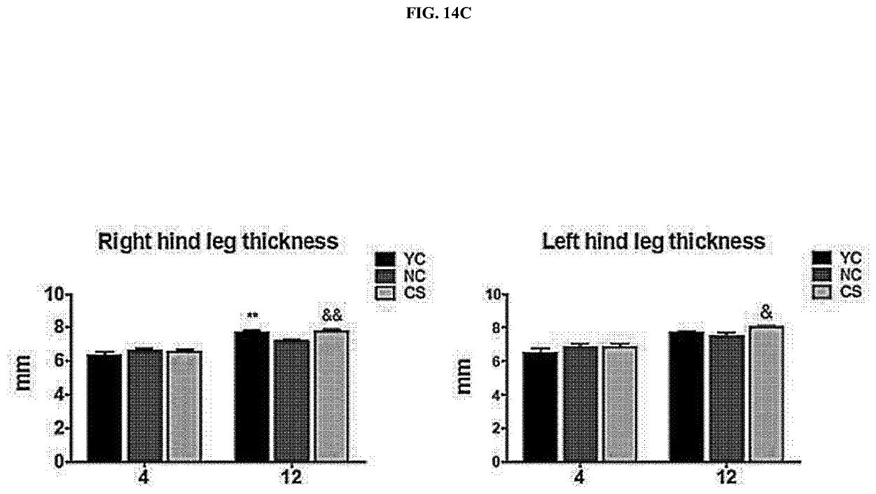

Changes in weight and strength of muscle tissue per 100 g of body weight are shown in FIGS. 14a and 14b. After the experiment was finished, the gastrocnemius muscle, femoral muscle, and tibialis anterior muscle of the experimental animals were excised and measured. As shown in FIG. 14a, the weights of the gastrocnemius muscle, tibialis anterior muscle, and total muscle tissue in the NC group significantly decreased compared to the YC group. In the CS group, gastrocnemius muscle and total muscle mass decreased by aging were significantly increased compared to the NC group.

As a result of the tensile strength measurement performed at the 12th week of the experiment, as shown in FIG. 14b, the tensile strength of the NC group was significantly reduced compared to that of the YC group, while the reduced tensile strength of the CS group was normalized to the level of the YC group.

As a result of measuring the thigh thickness of the hind limbs during the experimental diet feeding period, as shown in FIG. 14c, no significance was observed in all groups at the 4th week of feeding the experimental diet, however, at the 12th week of feeding the experimental diet, the thigh thicknesses of the right and left hind limbs of the CS group were all found to be significantly increased compared to the NC group.

### 5. Effect of Celery Seed Extract on Muscle Tissue Lipid Content

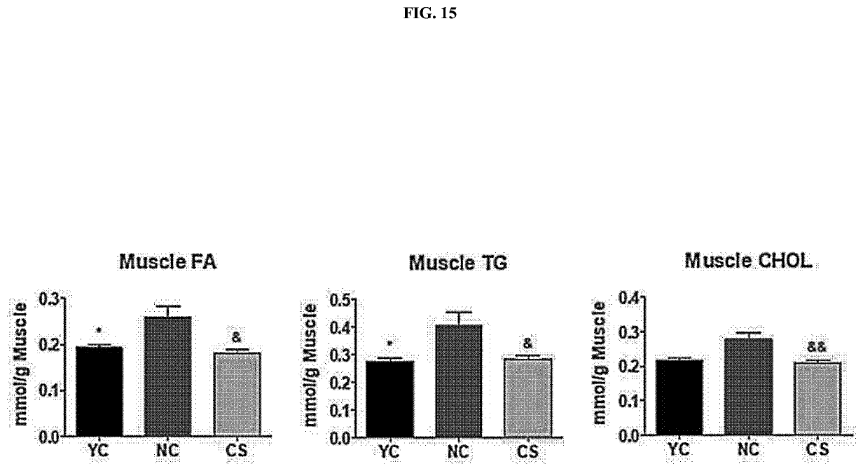

As a result of comparing free fatty acid (FA), triglyceride (TG) and cholesterol (CHOL) contents per unit weight of muscle tissue, as shown in FIG. 15, the neutral fat, free fatty acid and cholesterol contents of muscle tissue were significantly increased in the NC group. Further, with supplementation of celery seed extract, it was significantly reduced in the CS group compared to the NC group.

### 6. Morphological Analysis of Muscle Tissue

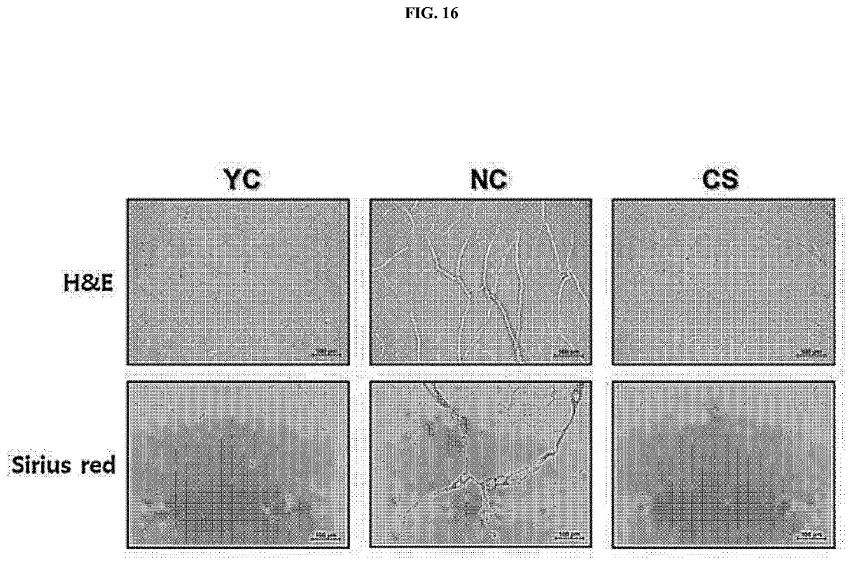

As a result of H&E staining for morphological analysis of the gastrocnemius muscle, as shown in FIG. 16, the shape and structure of the muscle cell bundle appeared irregular in the NC group compared to the YC group, with the supplementation of celery seed extract, it could be seen that the shape and structure described above were improved to normal levels. For histological visualization of collagen fibers in muscle tissue, Sirius red staining was performed. The collagen stained in the muscle tissue was clearly observed in the NC group compared to the YC group, it could be further confirmed that fibrosis of the muscle tissue in the CS group was inhibited by the supplementation of the celery seed extract in the CS group.

### 7. Immunochemical Analysis of Muscle Tissue

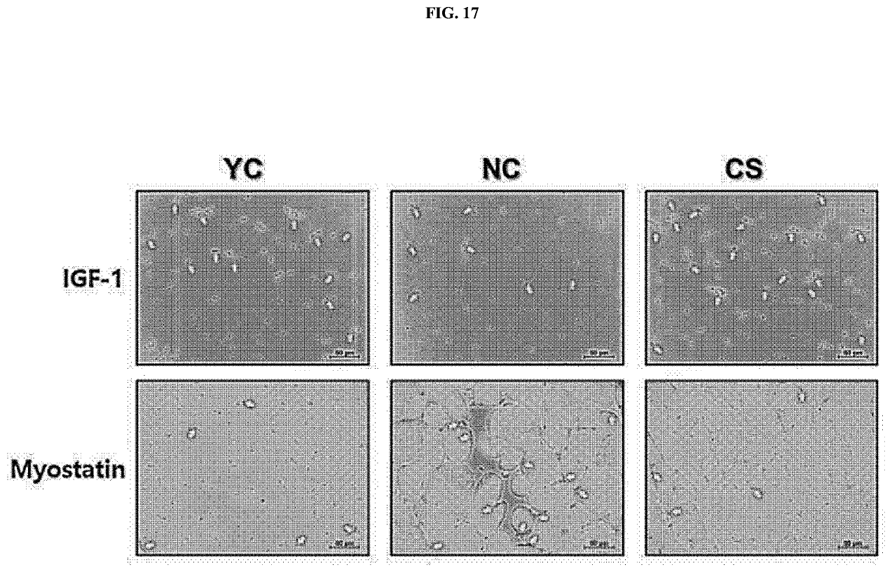

As a result of confirming the effect of celery seed extract on the expression levels of IGF-1 and Myostatin, which are involved in the synthesis and degradation of muscle tissue, as shown in FIG. 17, it was confirmed that IGF-1 is expressed lowly between cells in the muscle tissue of the NC group compared to the YC group, whereas the reduced expression of IGF-1 was increased by the supplementation of celery seed extract.

Further, it was confirmed that the expression of Myostatin decreased to a level similar to that of the YC group by supplementing with celery seed extract.

### 8. Effect of Celery Seed Extract on Muscle Function-Related Gene Expression

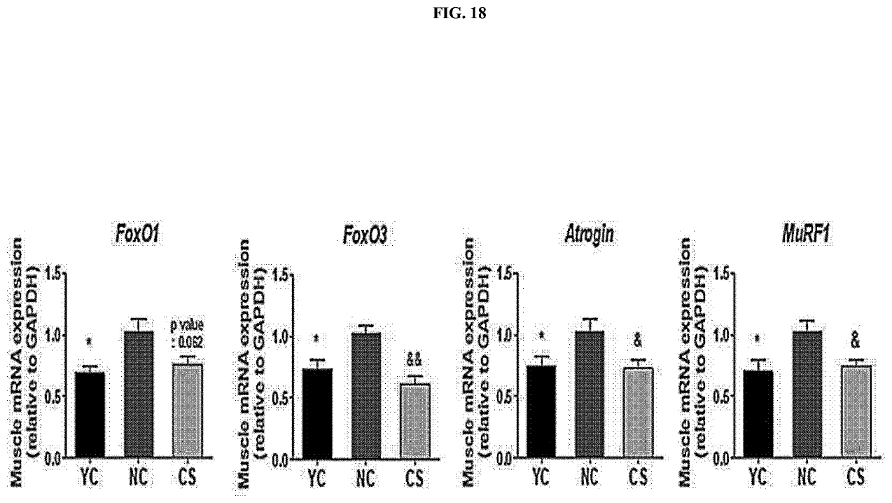

As a result of comparing the expression of muscle atrophy-related genes (FoxO1, FoxO3, Atrogin, and MuRF1) in the gastrocnemius muscle, as shown in FIG. 18, the expression of all muscle atrophy-related genes significantly increased in the NC group compared to the YC group. It was confirmed significant decrease by the supplementation of celery seed extract.

### 9. Effect of Celery Seed Extract on Muscle Fiber Protein Expression

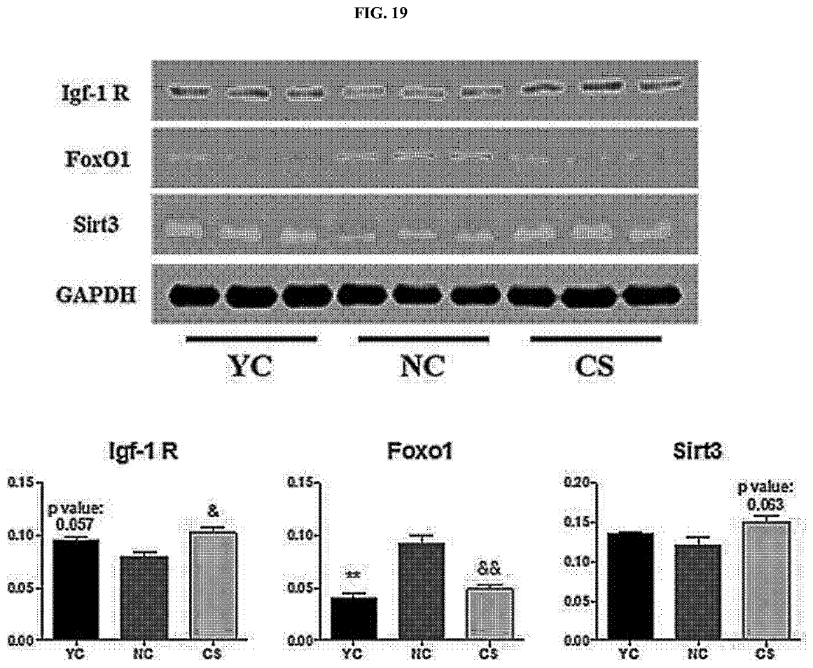

In order to identify the growth mechanism of muscle tissue cells by celery seed extract supplementation, the protein expression level of Igf-1 R pathway, which promotes muscle cell differentiation and is involved in muscle fiber protein accumulation, was measured. As a result, as shown in FIG. 19, the expression of Igf-1 R (Igf-1 Receptor) protein involved in muscle fiber protein synthesis was most strongly expressed in the CS group, whereas it was confirmed that the expression level decreased in the NC group. In the case of FoxO1, which is involved in inflammatory metabolism and muscular atrophy in muscle tissue, it was confirmed that it was most clearly expressed in the NC group compared to the YC group, and the expression decreased in the CS group.

Further, it was confirmed that the expression of Sirt3 protein, which regulates antioxidant metabolism in mitochondria, increased in the CS group compared to the NC group.

### 10. Intraperitoneal Glucose Tolerance Test

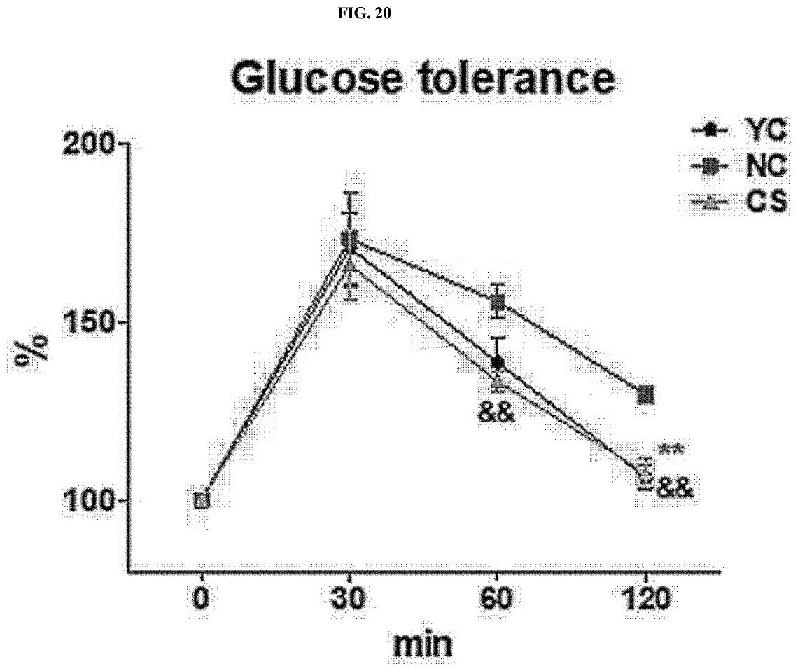

As a result of the glucose tolerance test performed at the 12th week of experimental diet feeding, as shown in FIG. 20, when 30 minutes elapsed after intraperitoneal glucose injection, the blood sugar in all groups increased, and after 120 minutes, it was confirmed that, compared to the NC group, the blood sugar of the YC group and the CS group was significantly decreased and showed a normal value.

### 11. Effect of Celery Seed Extract on Plasma Lipid Concentration

Table 5 shows plasma total cholesterol and other lipid concentrations measured in plasma obtained by sacrificing mice after feeding the experimental diet for 12 weeks. As shown in Table 5, total plasma cholesterol concentration and non-HDL cholesterol concentration in the CS group were significantly reduced compared to the NC group. On the other hand, HDL cholesterol concentration and the ratio of HDL cholesterol to total cholesterol (HDL-C/Total C ratio; HTR) were significantly increased in the CS group compared to the NC group.

TABLE 5

| | Experimental groups | | |
|---|---|---|---|
| Measurements | YC | NC | CS |
| Free fatty acid | 0.73 ± 0.03 | 0.76 ± 0.04 | 0.69 ± 0.05 |
| (mmol/L) | | | |
| Triglycerides | 0.93 ± 0.06 | 1.01 ± 0.15 | 1.00 ± 0.11 |
| (mmol/L) | | | |
| Total cholesterol | 4.80 ± 0.08 *** | 5.71 ± 0.11 | 4.85 ± 0.30 ^{&} |
| (mmol/L) | | | |
| HDL-cholesterol | 1.15 ± 0.03 | 1.18 ± 0.07 | 1.23 ± 0.07 |
| (mmol/L) | | | |
| nonHDL- | 3.68 ± 0.08 ** | 4.96 ± 0.26 | 3.93 ± 0.17 ^{&} |
| cholesterol | | | |
| (mmol/L) | | | |
| HTR (%) | 23.36 ± 0.65 * | 19.92 ± 1.16 | 25.61 ± 1.22 ^{&} |

### 12. Effects of Celery Seed Extract on Liver Tissue Weight, Liver Toxicity Index and Lipid Content

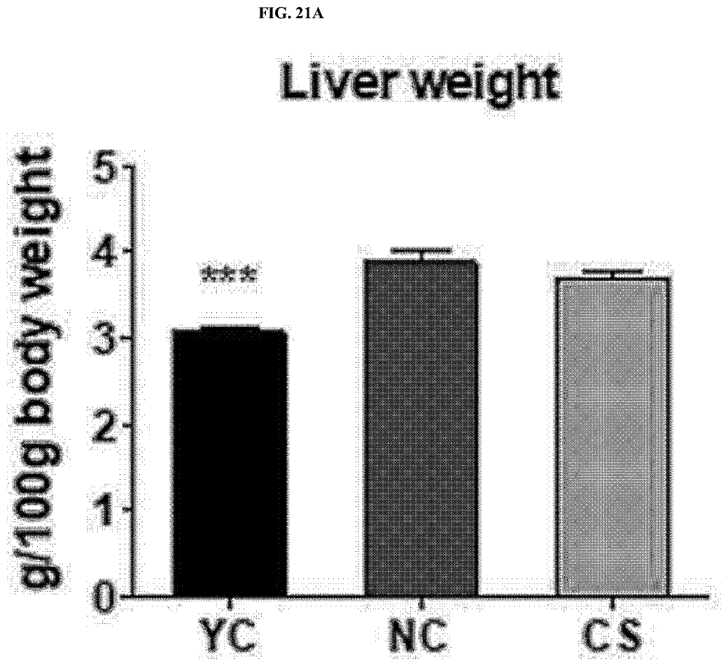

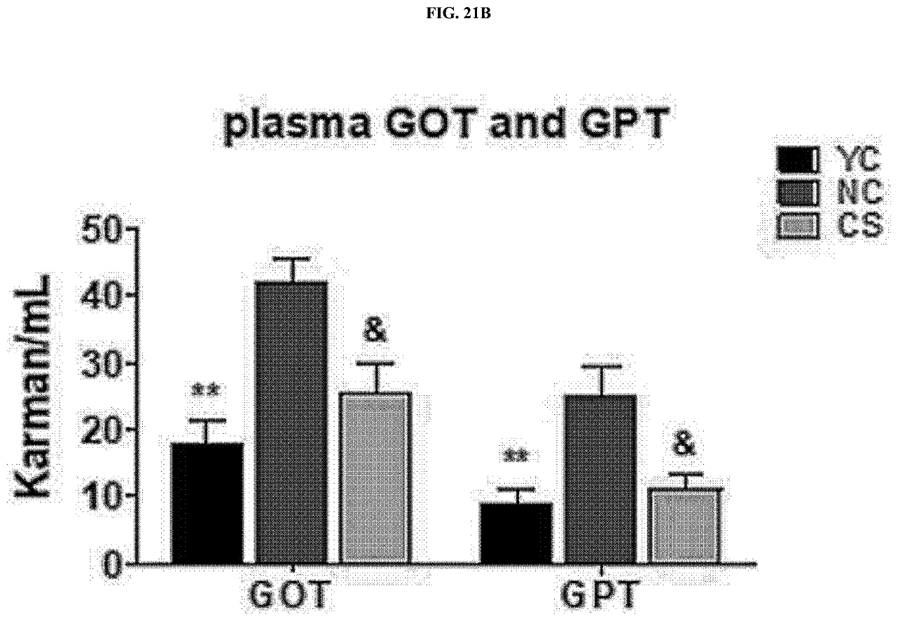

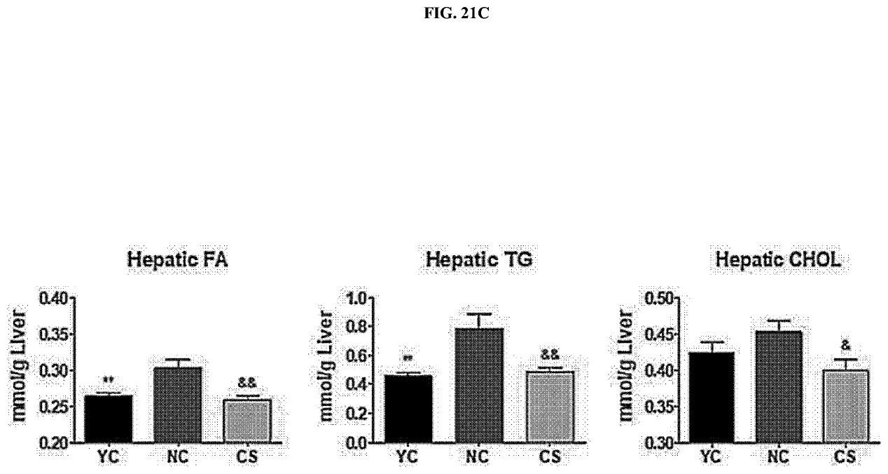

As for the weight of liver tissue per 100 g of body weight, as shown in FIG. 21a, hypertrophy was observed in the NC group compared to the YC group.

GOT and GPT, indicators of liver toxicity, are enzymes present in hepatocytes, and high levels of GOT and GPT in plasma generally indicate liver damage. As shown in FIG. 21B,[plasma GOT and GPT levels were significantly increased in the NC group compared to the YC group, and significantly decreased by the supplementation of celery seed extract.

Further, as shown in FIG. 21c, it was confirmed that the liver tissue lipid content of the NC group was significantly increased compared to the YC group, whereas the liver tissue lipid content of the CS group was significantly decreased compared to the NC group.

### 13. Antioxidant Related Biomarkers

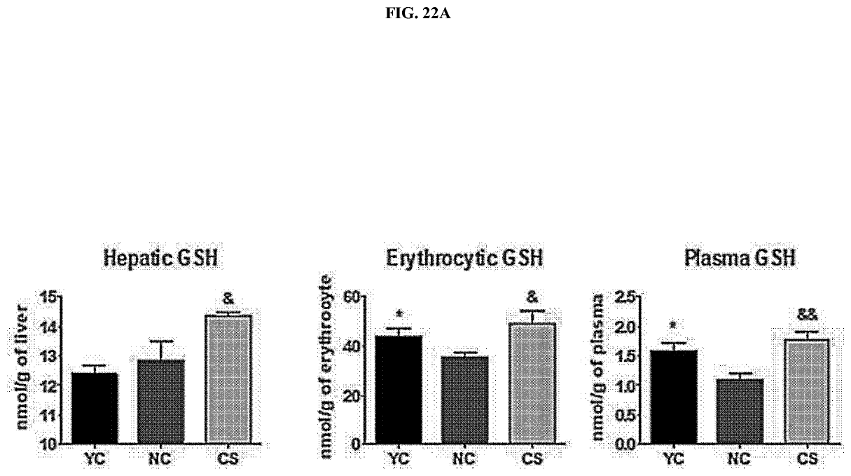

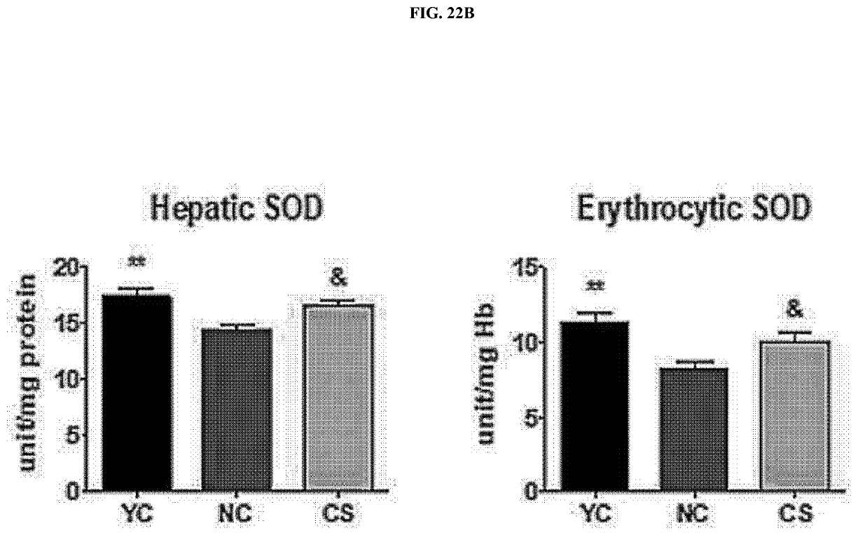

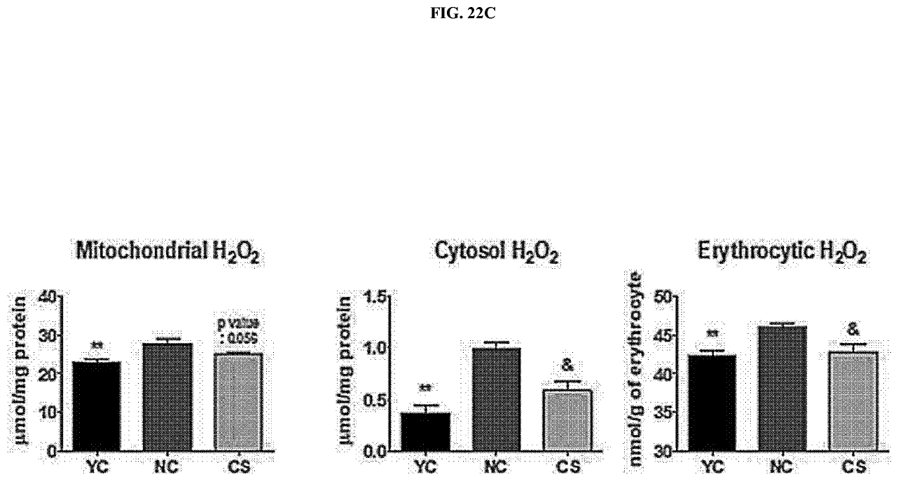

As a result of comparing the GSH content, which is an antioxidant-related biomarker, as shown in FIG. 22a, it was confirmed that the GSH content of liver tissue, red blood cells and plasma decreased in the NC group, but increased to the level of the YC group by supplementing with celery seed extract.

Further, SOD metabolic activity in liver tissue and erythrocytes was significantly reduced in the NC group compared to the YC group, as shown in FIG. 22b, but significantly increased by supplementing with celery seed extract.

As shown in FIG. 22c, the contents of liver tissue (mitochondria and cytosol) and erythrocytes H₂O₂ were significantly increased in the NC group compared to the YC group, and significantly decreased by the supplementing with celery seed extract.

### 14. Intestinal Microbiome Analysis

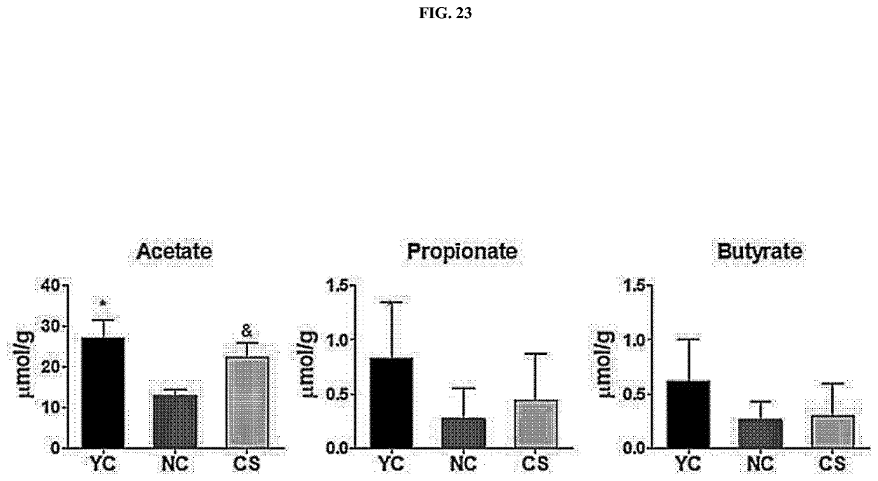

FIG. 23 shows the results of comparative analysis on short-chain fatty acid content and microbial flora using live mouse feces collected at 11 to 12 weeks of the breeding period. As a result of the short-chain fatty acid analysis, as shown in FIG. 23, the YC group showed a higher value than the NC group, and in particular, it was confirmed that a significant difference was shown in acetate. Further, it was found that supplementation of celery seed extract normalized the content of short-chain fatty acids decreased due to aging to the level of the YC group.

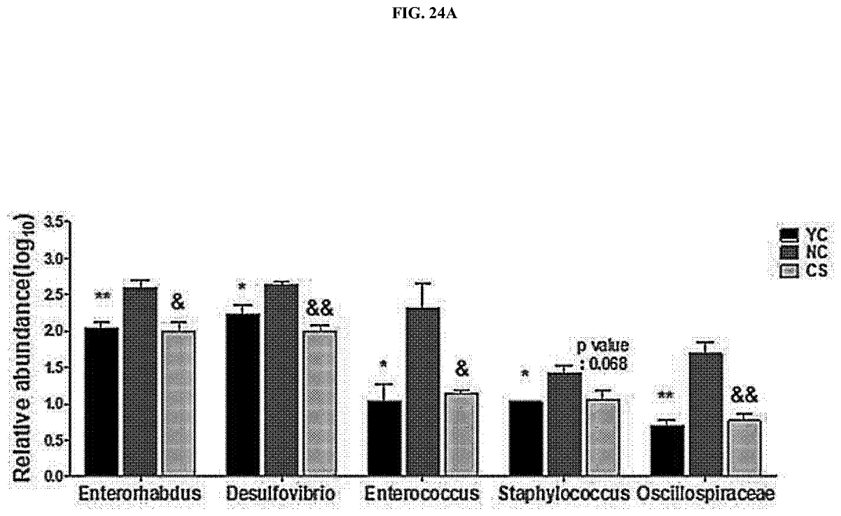

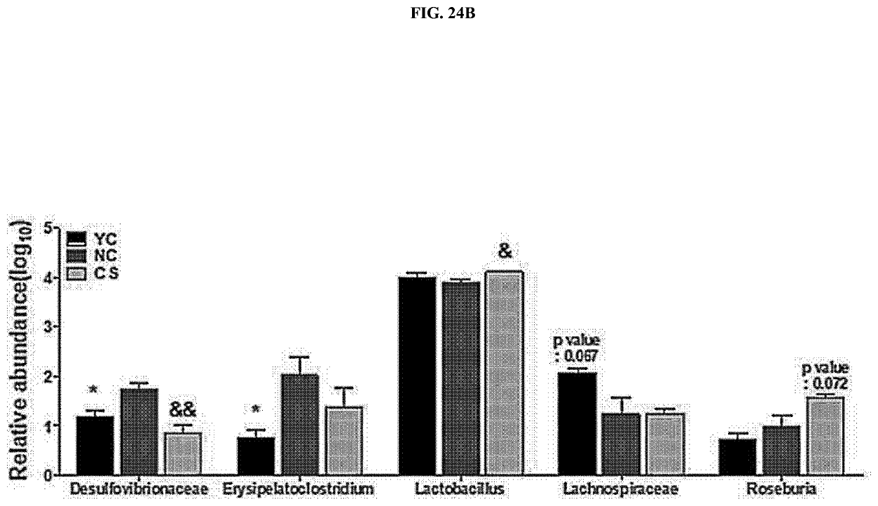

Intestinal microflora analysis showed the results analyzed at the genus stage. As a result of analyzing the microorganisms associated with the occurrence of inflammatory bowel disease and colitis, as shown in FIG. 24a, it was confirmed that the increased number due to aging with the supplement of celery seed extract was normalized to the level of the YC group. Further, as shown in FIG. 24b, microorganisms related to intestinal microbial imbalance (Desulfovibrionaceae family, Erysipelatoclostridium genus) were significantly increased in the NC group compared to the YC group, while being significantly decreased in the CS group compared to the NC group. Obesity/metabolic disease-related microorganisms (Lactobacillus genus) were significantly increased in the CS group compared to the NC group. As a result of the analysis of microorganisms involved in the synthesis of short-chain fatty acids, Lachnospiraceae, which is involved in production of acetate, tended to decrease in the NC group compared to the YC group, while Roseburia with supplementing with celery seed extract showed an increasing trend.

The above description of the present invention is for illustrative purposes, and those skilled in the art can understand that this will be easily modified into other specific forms without changing the technical spirit or essential features of the present invention. Therefore, the embodiments described above should be understood as illustrative in all respects but not limited thereto.

### INDUSTRIAL APPLICABILITY

The celery seed extract according to the present invention has no or few side effects using a natural product and can increase muscle mass and strength, so that it not only prevents muscle weakness but also suppresses the increase in body weight and body fat, and reduces blood lipid concentration and liver toxicity index (GOT And GPT), whereby it can be usefully applied to prevent, improve, or treat muscle weakness-related diseases, metabolic diseases, or liver diseases, thereby attaining industrial applicability.

1-16. (canceled)

17. A method for prevention or treatment of muscle weakness-related diseases, comprising administering a composition including celery seed extract as an active ingredient.

18. The method of claim 17, wherein the composition is pharmaceutical or food composition.

19. The method of claim 17, wherein the celery seed extract is an extract obtained by one or more solvents selected from the group consisting of water, alcohol having 1 to 6 carbon atoms, acetone, ether, benzene, chloroform, ethyl acetate, methylene chloride, hexane, cyclohexane, petroleum ether, subcritical fluid, and supercritical fluid.

20. The method of claim 17, wherein the muscle weakness-related disease is one or more selected from the group consisting of sarcopenia, muscular atrophy, muscle dystrophy, and cardiac atrophy.

21. The method of claim 20, wherein the sarcopenia is age-related sarcopenia or obesity sarcopenia.

22. The method of claim 17, wherein the composition has one or more of the following effects:
(a) increase in muscle mass;
(b) inhibition of muscle mass loss;
(c) increased muscle strength;
(d) inhibition of increase muscle tissue lipid content; or
(e) inhibition of muscular fibrosis.

23. The method of claim 22, wherein the composition has one or more of the following effects:
(a) inhibition of body weight and body fat gain;
(b) inhibition of fibrosis in liver or adipose tissue;
(c) inhibition of plasma lipid concentration increase;
(d) inhibition of increase in liver tissue lipid content;
(e) inhibition of increase in plasma hepatotoxicity index;
(f) inhibition of lipid peroxide increase and increase of antioxidant activity; or
(g) improvement of intestinal microbial imbalance.

24. A feed or feed additive for reinforcing muscle strength, comprising celery seed extract as an active ingredient.

25. A use of a composition comprising celery seed extract, for preventing or treating muscle weakness-related diseases.

26. A use of celery seed extract for production of a drug for treating muscle weakness-related diseases.
